(19) United States
(12) Patent Application Publication
(10) Pub. No.: US 2012/0165760 A1
(43) Pub. Date: Jun. 28, 2012

(54) DRUG ELUTING IMPLANT

(75) Inventors: Jeffrey J. Missling, Eden Prairie, MN (US); Bryan A. Claseman, Buffalo, MN (US)

(73) Assignee: SurModics, Inc., Eden Prairie, MN (US)

(21) Appl. No.: 13/338,695

(22) Filed: Dec. 28, 2011

Related U.S. Application Data
(60) Provisional application No. 61/427,628, filed on Dec. 28, 2010.

(51) Int. Cl. A61M 5/00; B05D 7/00

(52) U.S. Cl. 604/264; 604/93.01; 427/2.24

(57) ABSTRACT

Described herein is an implantable medical device that has one or more surfaces that include one or more shallow recesses configured to receive a drug delivery composition comprising a polymeric matrix and one or more biologically active agents. In one embodiment, one or more surfaces comprise a frictional surface. In a more particular embodiment, the implantable medical device comprises a stem configured for insertion into a bone of a patient, wherein the stem comprises one or more surfaces that include one or more shallow recesses configured to receive a drug delivery composition comprising a matrix and one or more biologically active agents. The drug delivery composition can be a durable drug delivery matrix, a biodegradable drug delivery matrix, or a combination thereof. Methods of making such an implant are also provided.

This application claims the benefit of U.S. Provisional Application No. 61/427,628, filed Dec. 28, 2010, the contents of which are herein incorporated by reference.

### FIELD OF THE INVENTION

This invention relates generally to the field of medical implants. In particular, the invention relates to a drug eluting implant.

### BACKGROUND OF THE INVENTION

Patients who suffer from joint pain and immobility, for example, caused by osteoarthritis and/or rheumatoid arthritis have an option of joint replacement surgery in which the diseased and/or damaged joint is replaced with a prosthetic joint. Joint replacement surgery, otherwise known as joint arthroplasty, enables many individuals to function properly when it would not be otherwise possible to do so. In a typical total joint arthroplasty, the ends or distal portions of the bones adjacent to the joint are resected or a portion of the distal part of the bone is removed and the artificial joint is secured thereto. Examples of artificial joints include elbows, hips, knees and shoulders, which are typically made of metal, ceramic and/or plastic components that are fixed to existing bone.

One problem encountered with the use of implantable medical devices is the colonization of bacterial and fungal organisms on one or more surfaces of the implant. Another difficulty is the need to avoid an adverse immune response. As such, an implant from which one or more bioactive agents can elute may be desirable. Various attempts have been made to prepare a drug eluting orthopedic implant. One method is to coat one or more surfaces of an implanted medical device with an antibiotic. However, the efficacy of the bioactive agent that is coated on the surface of an orthopedic implant is limited. For example, after the device is implanted, the bioactive agent can quickly leach from the surface of the device into the surrounding environment, such that the amount of bioactive agent present on the surface decreases to a point where the protection against bacterial and fungal organisms is no longer effective. One solution to the leaching problem is to coat the implant with a drug delivery matrix. However, the drug delivery coating tends to delaminate from the device due to exposure to frictional forces during implantation.

Accordingly, there is a need for a drug eluting medical device, for example an orthopedic implant, which can be exposed to frictional forces without destruction or loss of efficacy.

### SUMMARY

Described herein is an implantable medical device that has one or more surfaces that include one or more shallow recesses configured to receive a drug delivery composition comprising a matrix and one or more biologically active agents. In one embodiment, the recess has a maximum depth that is less than the length of the recess. In another embodiment, the maximum depth of the recess is greater than the width of the recess. In yet another embodiment, the maximum depth of the recess is less than the width of the recess. In another embodiment, the depth of the recess is less than both the width and the length of the recess.

In one embodiment, one or more surfaces comprise a frictional surface. In a more particular embodiment, the implantable medical device comprises a stem configured for insertion into a bone of a patient, wherein the stem comprises one or more surfaces that include one or more shallow recesses configured to receive a drug delivery composition comprising a matrix and one or more biologically active agents. In a more particular embodiment, the implantable medical device is an orthopedic implant adapted to be implanted at a site of interest within a bone or joint, for example, a prosthetic selected from a hip, shoulder, knee, elbow, ankle, wrist, and finger prosthetic. In other embodiments, the implantable medical device is a fixation device, for example, a pin, screw, or rod. In still other embodiments, the implantable medical device is a spinal or dental implant.

Methods of making such an implant are also provided. In general, the method includes steps of: obtaining a device that comprises one or more frictional surfaces; forming one or more shallow recesses on one or more frictional surfaces; and introducing a drug delivery composition into one or more shallow recesses.

The above summary of the present invention is not intended to describe each discussed embodiment of the present invention. This is the purpose of the figures and the detailed description that follows.

### DRAWINGS

The invention may be more completely understood in connection with the following drawings, in which:

FIG. 1 is an illustration of a ball and socket joint;

FIG. 2 is an illustration of a prosthetic knee joint;

FIGS. 3A and B are an illustration of a prosthetic elbow joint;

FIG. 4 is an illustration of a bone screw;

FIGS. 5A and B are a partial view of an implantable medical device with a shallow recess;

FIGS. 6A-D are illustrations of an orthopedic rod with various shallow recess configurations;

FIG. 7 is a partial illustration of a bone screw;

FIG. 8 is a close up illustration of a myocardial screw;

FIG. 9 is an illustration of a dental implant;

FIG. 10 is an illustration of a stent and catheter; and

FIGS. 11A-C are illustrations of various configurations of shallow recesses on a myocardial screw.

While the invention is susceptible to various modifications and alternative forms, specifics thereof have been shown by way of example and drawings, and will be described in detail. It should be understood, however, that the invention is not limited to the particular embodiments described. On the contrary, the intention is to cover modifications, equivalents, and alternatives falling within the spirit and scope of the invention.

### DETAILED DESCRIPTION

The invention described herein relates to an implantable medical device. In one embodiment, the invention relates to a drug eluting implantable medical device. In particular, the invention relates to a drug eluting implant on which one or more surfaces encounters frictional force during implantation and/or use. The term “friction” or “frictional force” refers to the force that resists the relative motion between surfaces in contact with each other. The term “frictional surface” refers to a surface that encounters frictional force. Frictional surfaces can be found within an implant (for example, in a implant with one or more moving parts, such as a femoral head and liner found in a prosthetic hip); between the surfaces of two or more implants (for example, between the internal surface of a stent and the external surface of a catheter used for implantation); or between an implant and a tissue of a patient (for example, at the bone-implant interface of an orthopedic implant).

In general, the invention provides an implantable medical device in which a drug delivery matrix is localized within one or more grooves or recesses on one or more surfaces of the device. In one embodiment, the implantable medical device includes one or more frictional surfaces and a drug delivery matrix, wherein at least some of the drug delivery matrix is localized within one or more grooves or recesses on one or more frictional surfaces of the device. It is believed that localizing the drug delivery matrix within one or more grooves or recesses on a frictional surface of the device will reduce the likelihood of delamination of the drug delivery matrix, since the drug delivery matrix will be protected from shear forces encountered by the frictional surface.

Implantable Medical Device

The invention described herein is suitable for use in connection with a wide variety of implantable medical devices, either permanent or temporary. The implant can be constructed from any suitable material. In one embodiment, the material is biocompatible. As used herein, the term “biocompatible” means that the material causes little, if any, local or systemic problems due to an immune response or an allergic reaction. The device can be made from materials including, but not limited to, metals and metal alloys, such as titanium, titanium alloys, tantalum, tivanium, vitallium, chromium alloy, cobalt alloy, and stainless steel; sintered glass; artificial bone; ceramic; high-strength plastic, including, but not limited to high density polyethylene high and ultra high molecular weight polyethylene; or a combination thereof. In some embodiments, the implant may have one or more structures for anchoring the implant to a bone and/or one or more structures or porous surfaces to encourage bone ingrowth.

In general, the implant includes one or more frictional surfaces. In one embodiment, one or more frictional surfaces can be found between parts of the implant. In another embodiment, one or more frictional surfaces are located between the surfaces of two or more implants. In yet another embodiment, one or more frictional surfaces are located between an implant and one or more tissues of a patient. For the sake of brevity, the disclosure will focus on frictional surfaces that are located between an implant and one or more tissues of a patient. However, it is understood that the invention described herein is applicable to other types of implantable medical devices, in particular other medical devices with one or more frictional surfaces.

In one embodiment, the implant is a joint prosthesis. The term “joint” refers to an area where two bones are attached for the purpose of motion of body parts. An articulation, or joint, is usually formed of fibrous connective tissue and cartilage. Joints are grouped according to their motion: a ball and socket joint; a hinge joint; a condyloid joint (a joint that permits all forms of angular movement except axial rotation); a pivot joint; gliding joint; and a saddle joint. Examples of joints include, but are not limited to, joints found in the hip, shoulder, knee, elbow, ankle, wrist, and finger.

In one embodiment, the implantable medical device is an orthopedic implant that includes a stem that is inserted into a bone cavity. As used herein, the term “stem” refers to an elongate structure, usually configured to anchor the device to a tissue of a patient. In general, an orthopedic implant is used to replace a damaged joint. Many joints within the human body, including but not limited to hip and shoulder joints, have a similar geometry. Although the details may vary, these joints generally include: (a) a more-or-less spherical ball; (b) an attachment to a long bone; and (c) a hemispherical socket, wherein the ball is retained within the socket such that the long bone may pivot and articulate.

When a joint has been destroyed or damaged by disease or injury, the joint can be replaced with an artificial prosthesis. In general, a joint replacement can include one or more components that simulate a natural joint, for example: (a) a more-or-less spherical ball; (b) a “stem,” which is configured to be inserted within a medullary cavity or canal of a bone; and (c) a hemispherical socket configured to retain the spherical ball, such that the ball can rotate within the socket, allowing the joint to pivot and articulate. The medullary cavity of a bone is the central cavity of a shaft where red bone marrow and/or yellow bone marrow is stored. The term “intramedullary” refers to the inside of a bone.

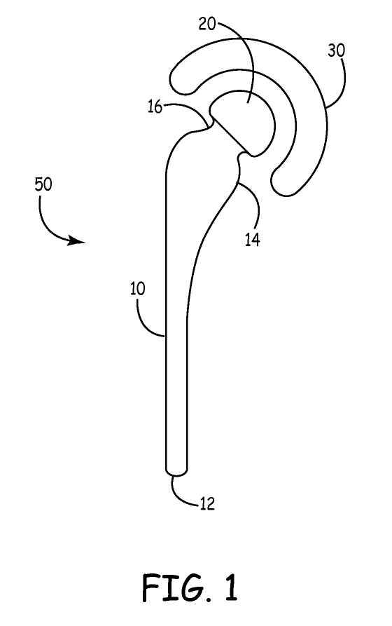

A schematic of a “ball and socket” orthopedic prosthesis is shown in FIG. 1. Generally, the prosthesis includes a stem 10; a more-or-less spherical ball 20; and a hemispherical socket 30. The stem 10 is generally configured for placement within a bore (not shown), for example, a medullary canal of a long bone and includes a distal end 12, a proximal end 14 with a neck 16 adapted to receive a ball 20. When in use, the stem 10 is placed within a medullary canal of a femur (not shown) such that proximal end 14 extends outwardly from the medullary canal of the femur to cooperate with the hemispherical socket 30 by means of the ball 20. In one embodiment, the stem 10 and ball 20 are one piece. In other embodiments, the stem 10 and ball 20 are provided separately, resulting a modular device that allows for additional customization. Furthermore, each part of the device can be provided in a variety of sizes to accommodate various body sizes and types.

The stem 10 of the device can be secured within the bore by any suitable means, including but not limited to the use of an adhesive or cement, or friction.

A friction fit can be achieved with either a positive or negative allowance. As used herein, the term “allowance” refers to the difference between the size of the bore and the size of the stem. A “positive allowance” refers to a configuration in which the size of the bore is larger than the stem. A “negative allowance” refers to a configuration in which the size of the bore is smaller than the stem. As used herein, the term “size” generally refers to the outside diameter of the stem and the inside diameter of the bore. When there is a negative allowance, the stem is secured within the bore by friction after the parts are coupled. This is sometimes referred to as an interference fit, a shrink fit or press fit. Depending upon the allowance, a stem in a configuration with a positive allowance can be secured to the bore by friction or by the use of a cement or adhesive. Because the surface of the stem is exposed to frictional forces during implantation, a drug delivery matrix coated on the frictional surface of the stem can delaminate during implantation. As such, it may be desirable to include a drug delivery composition within one or more shallow recesses on a surface of the stem, to reduce the likelihood of delamination.

If desired, the prosthetic can be affixed using a cemented or an uncemented system. In other embodiments, the device can include a surface coating or texture to stimulate bone remodelling and/or to help secure the implant.

Cemented stems use acrylic bone cement to secure the stem to the bone. Bone cement typically includes methylmethacrylate (MMA) monomer and polymethylmethacrylate (PMMA) powder. Commercially available bone cements can differ from each other in particle size and composition of the PMMA powder, concentrations of accelerators such as N,N-dimethyl-p-toluidine, or the use of special additives such a radiopaque or X-ray contrast agents, antibiotics, and dyes.

In general, the implantation process for the prosthetic is as follows. First, an incision is made to access the joint and prepare the implant site. The hemispherical cup is then secured within the hemispherical socket. The hollow shaft of the long bone is cleaned and enlarged, creating a cavity sized to receive the implant stem. The proximal end of the long bone may be planed and smoothed so the stem can be inserted flush with the bone surface. Once the size and shape of the bone surfaces are satisfactory, the stem is inserted into the canal. During insertion, the surface of the stem can be exposed to significant frictional forces. If the ball is a separate component, the proper size is selected and secured to the stem. Finally, the ball is seated within the cup so the joint is properly aligned and the incision is closed.

Ball and Socket Joints

The hip joint is called a ball-and-socket joint because the spherical head of the femur moves inside a cup-shaped acetabulum of the pelvis. A hip replacement implant can include one or more of the following: a stem 10 configured for placement within the hollow shaft of the femur; a ball 20 configured to replace the spherical head of the femur; and a hemispherical cup 30 to replace the acetabular hip socket.

Although the size and shape of the components differ, a shoulder replacement prosthetic and the associated implantation process is somewhat similar to the hip replacement prosthetic and implantation process. As with hip replacement surgery, shoulder replacement involves replacing the shoulder joint with an implant that includes a ball 20 attached to a stem 10, and an artificial socket 30.

Hinge Joints

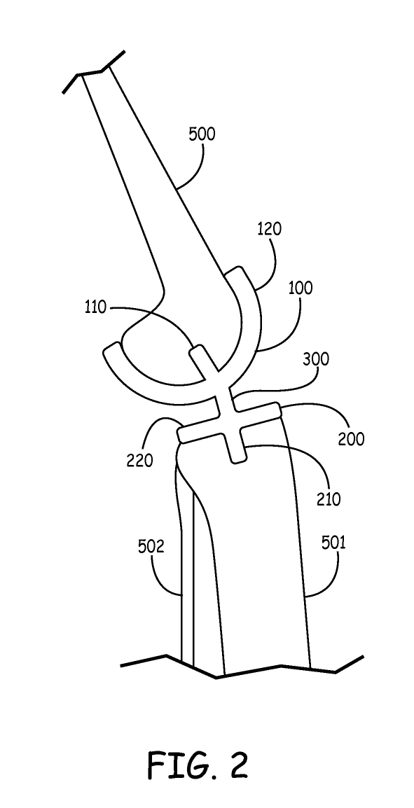

The knee joint is where the lower end of the femur 500 meets the upper end of the tibia 501. The patella sits in front of the joint to provide protection. An illustration of a knee implant is shown in FIG. 2 and includes one or more of the following components: a femoral component 100 with a condyloid portion 120 and a fixation stem 110; a tibial component 200, which includes a weight-bearing portion 220 simulating the plateau of the tibia 501 and a fixation stem 220; a shaft 300 between the femoral 100 and tibial 200 prosthesis which is load or weight-bearing; and a patellar component (not shown), which is typically a dome-shaped element that duplicates the shape of the patella.

A wide variety of configurations for elbow prosthetics are known. One type of elbow prosthesis is a linked or constrained elbow prosthesis, which includes a simple hinge arrangement, in which one component is attached to the end of the humerus and the other component is attached to the end of the ulna.

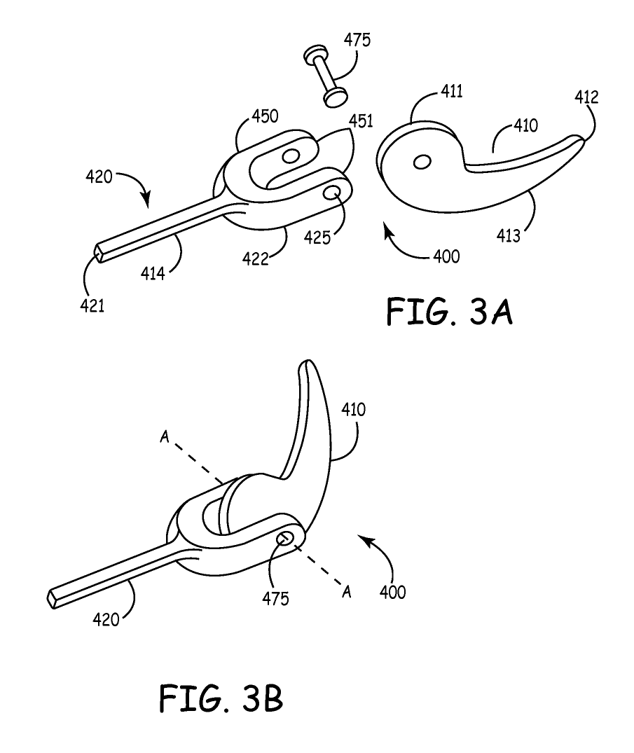

An illustration of one embodiment of a linked elbow prosthetic for replacement of an elbow joint is shown generally at FIGS. 3A (exploded) and 3B (linked). In general, an elbow joint prosethetic includes a humeral component 420 and an ulnar component 410. The humeral component 420 includes a first stem 414 located at a proximal end 421, wherein the stem 414 is adapted to fit within the meduallary canal of a humerus (not shown). The humeral component 420 also includes a distal portion 422 with a generally U-shaped portion 450 that has two arms 451. An aperture 425 extends perpendicularly through each of the 451.

The ulnar component 410 includes a second stem 413 located at a distal end 412 which is adapted to fit within the medullary canal of an ulna (not shown). The ulnar component 410 also includes a proximal end 411 which is configured to be coupled to the U-shaped portion 450 of the humeral component 420. FIG. 3B shows the humeral 420 and ulnar 410 components coupled using a fastener 475, such as a hinge pin, such that the humeral 420 and ulnar 410 components are rotatable about the axis A-A.

A prosthetic ankle joint (not shown) is similar to an elbow prosthesis. In general, an ankle prosthesis includes a tibial component that has a first stem that is configured to be inserted into the tibia and a talus component that has a second stem that is configured for insertion into the talus, wherein the tibial and talus component are moveably coupled to allow the relative movement between the tibial and talus components.

A prosthetic wrist joint (not shown) similarly includes a radial component and a metacarpal component. The radial component has a stem for insertion into the end of the radius bone and a cylindrical component. The metacarpal component has a hinge portion and anchoring component for anchoring the device to one or more metacarpals (e.g., to the second and third metacarpal) and ball portion. The prosthetic wrist joint may also include an intermediate component includes a cylindrical bearing surface configured to engage the cylindrical surface of the radial component, and a recess for receiving the ball portion of the metacarpal component. The ball and socket arrangement between the metacarpal component and the intermediate component permit rotational movement of the joint.

A finger prosthetic (not shown) similarly includes one or more of the following three components: a metacarpal component, a phalangeal component, and a hinge. All three components operate in cooperation to provide twisting, flexing, pistoning, and lateral motions simulating the human finger joint.

Internal Fixation Devices

The invention can also be used in connection with internal fixation devices, for example, devices used to bring fractured bone surfaces into close contact or to secure an implantable medical device to a tissue of a patient. Examples of internal fixation devices include, but are not limited to, pins, nails, rods and screws. One or more surfaces of an internal fixation device may be exposed to frictional forces during implantation and/or use. As such, it may be desirable to include a drug delivery composition within one or more shallow recesses on one or more surfaces of an internal fixation device.

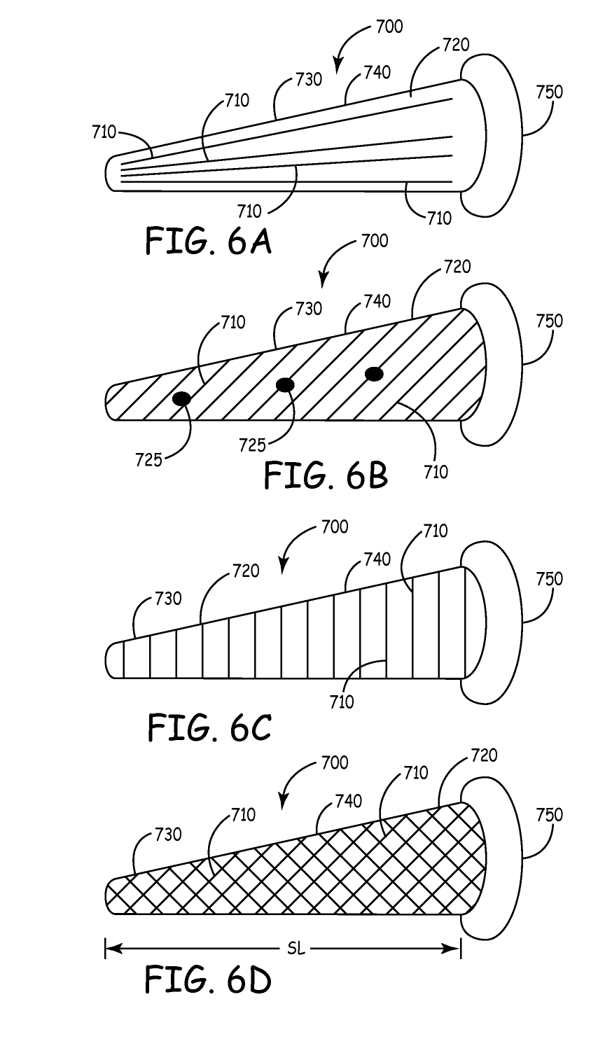

Orthopedic pins, nails and rods serve the same general purpose—to fix bone fractures. However, they can vary in size and mode of implantation. In general, a rod or nail is used to fix a fracture of a long bone, for example, by axial insertion of the rod or nail through the medullary canal of the bone. As shown in FIG. 6, a nail or rod 700 generally has an elongated stem 740 (or proximal portion) and a transversely flattened head 750 (or distal portion). The elongate stem 740 may define a plurality of transverse apertures 725 for receiving one or more bone screws (not shown). In use, the stem 740 of the nail or rod 700 is advanced axially through the hollow medullary canal and screws are applied through the transverse apertures on both sides of the fracture, thus securing the bone into a single, immoveable piece and allowing healing to take place along the fracture site. The elongate stem 740 may be hollow or solid.

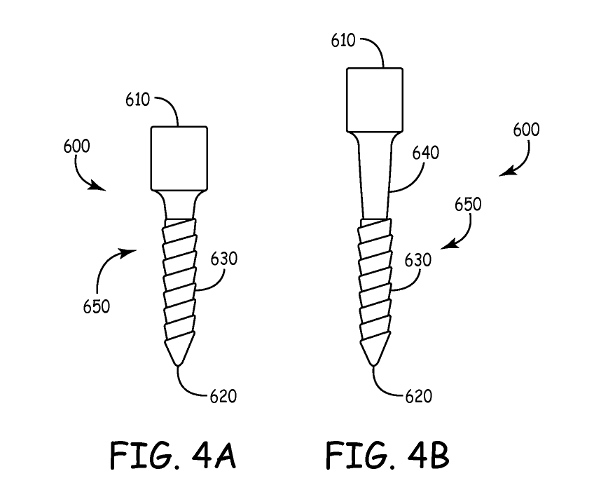

In another embodiment, the implantable medical device is a bone screw. The term “bone screw” refers to an internal fixation device used to hold together two pieces of bone together that moves linearly as it is rotated. Bone screws can be used, for example, to fasten two vertebrae of a human spinal column relative to one another. A variety of bone screw configurations exist and can be used in connection with the invention described herein. A schematic of a bone screw 600 is shown in FIGS. 4A and 4B. In general, a bone screw 600 includes a distal screw head 610, a proximal tip 620, an elongate stem 650, which includes threads 630, the helical structure used to convert rotational movement to linear movement. In one embodiment, the threading is present at the leading end of the stem 650 and the head 610 at the trailing end is separated from the thread 630 by a smooth, cylindrical shank 640 (See, FIG. 4B). In another embodiment, the stem 650 of the bone screw 600 is threaded over its full length (See, FIG. 4A).

Another example of an internal fixation device is a spinal fixation device (not shown). A typical spinal fixation system includes bone screws that are installed in the vertebrae of the spinal column and a rigid plate or rod that is secured to the screws.

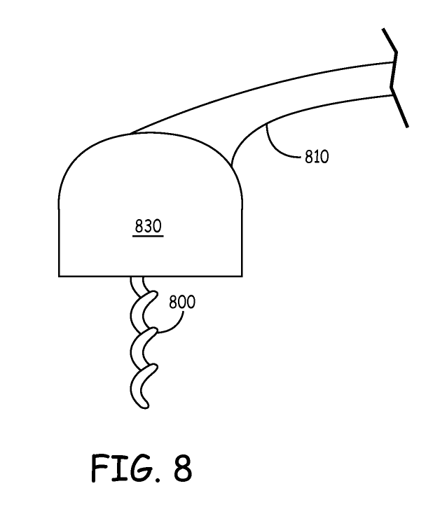

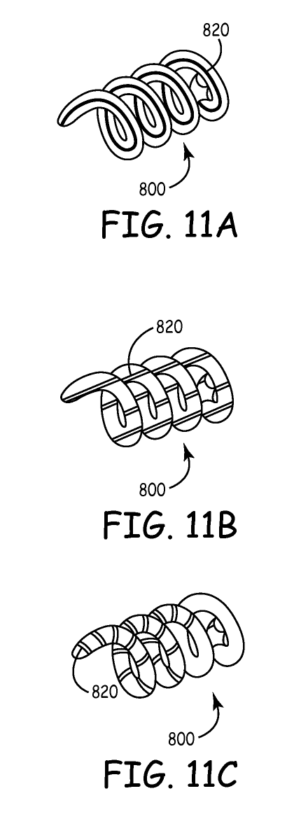

FIG. 8 shows another example of an internal fixation device, a myocardial screw 800. Briefly, a myocardial screw 800 is a helical fixation element typically used to secure a header body 830 and lead 810 of a cardiac stimulation and/or sensing device to the heart of a patient. A shallow recess or groove can be placed along one or more surfaces of the helical myocardial screw 800 to deliver one or more bioactive agents to the patient. In one embodiment, one or more shallow recesses 820 are located along the length of the screw body 800 and can be helix parallel (e.g., parallel to the centerline of the helical wire, as shown in FIG. 11A); axial (e.g., parallel to the central axis of the helix body, as shown in FIG. 11B); and/or circumferential (e.g., perpendicular to the centerline of the wire forming the helix, as shown in FIG. 11 C). Other configurations are also possible.

Dental Implants

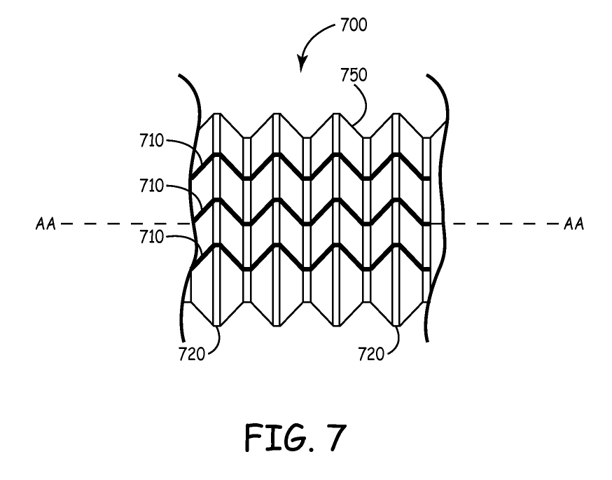

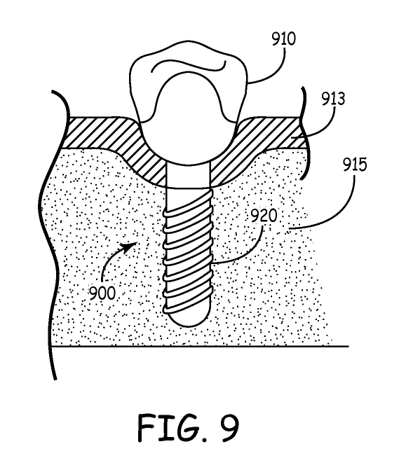

An illustration of a dental implant is shown in FIG. 9. In general, a dental implant is used to restore one or more teeth in a patient's mouth with a device that includes a stem 900 that functions similarly to the root structure for a natural tooth and a body 910, resembling a crown structure that is visible above the gum 913 of the patient. In one embodiment, the stem 900 includes threads 920 to secure the stem 910 to the jawbone 915. In another embodiment, the stem 900 does not include threads and is secured to the jawbone 915 by friction and/or using a bone cement. One or more recesses can be included on one or more frictional surfaces of the screw, as illustrated in FIG. 7.

Stents and Catheters

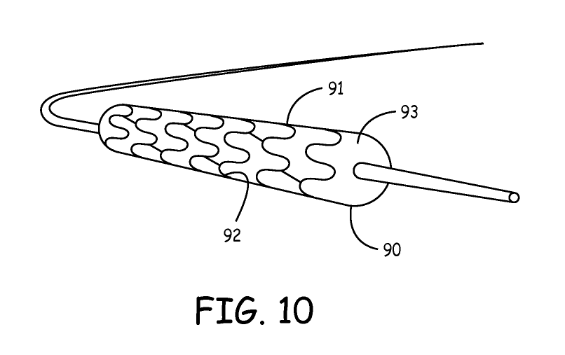

A stent is a generally cylindrical, flexible device that is placed within the lumen of a vessel to hold the lumen open, for example to prevent restenosis. One type of stent is expanded to the desired size at the desired site within the vessel by inflating a balloon catheter, referred to as “balloon-expandable” stents. One example of a balloon-expandable stent is shown in FIG. 10, in which a stent 91 is secured onto a surface 93 of a deflated balloon catheter 90. However, during delivery, the luminal surface 92 of the stent 91 may be exposed to frictional forces while the catheter system is advanced.

Various types of stents are available. In general, a stent 91 is a cylindrical metal mesh with an initial crimped outer diameter, which may be forcibly expanded by the balloon to a deployed diameter. When deployed in a body passageway of a patient, the stent may be designed to preferably press radially outward to hold the passageway open.

At times, it may be desirable to provide a stent that is able to deliver one or more biologically active agents to the patient, for example, anticoagulants, antiproliferatives, or antirestenosis compounds.

Shallow Groove or Recess

The invention relates to an implantable medical device that is used in combination with a drug delivery matrix to provide one or more bioactive agents to a patient. In one embodiment, the implantable medical device includes one or more frictional surfaces. In a more particular embodiment, the implantable medical device includes one or more frictional surfaces that include one or more shallow recesses configured to receive a drug delivery composition. The drug delivery matrix in the shallow recesses is protected from frictional forces applied to the surface, thus reducing the likelihood of damage or delamination to the drug delivery matrix. Additionally, the number, size and/or distribution the recesses can be varied based on therapeutic considerations, including, for example, target tissue and/or dosage.

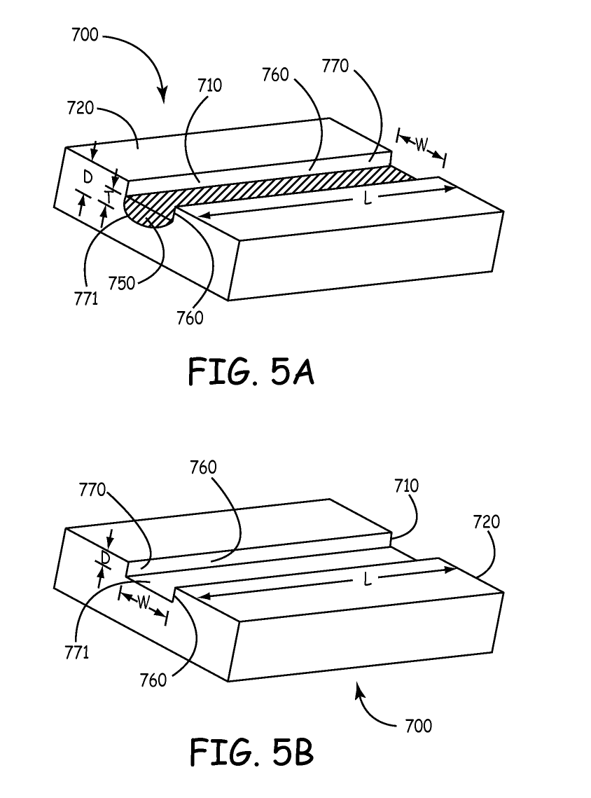

As used herein, the term “shallow recess” refers to a cavity or groove present on the surface of the device. FIGS. 5A and 5B show a close-up illustration of a shallow recess 710 in the surface 720 of part of a device 700. The recess 710 defines an opening in the surface 720 of the device that has two or more sidewalls 760 and a base 771, wherein the sidewalls 760 and base 771 define a cavity with a length (L), a depth (D) and a width (W). In one embodiment, shown in FIG. 5A, the depth (D) of the recess is not constant across the width (W) of the recess. Although the cross-section of the recess 710 shown in FIG. 5A is semi-circular, other configurations are also possible, for example, a recess 710 having a cross-sectional shape that is parabolic, polygonal, including, but not limited to triangular, rectangular, square, trapezoidal; irregular, or a combination thereof. In one embodiment, the cross sectional shape and/or size of the recess 710 is constant along the length of the recess. In another embodiment, the cross sectional shape and/or size varies along the length of the recess. As used herein, the term “constant” means that the dimensions are constant within an acceptable margin of error. In other embodiments, such as the example shown in FIG. 5B, the depth (D) of the recess is constant across the width (W). The term “maximum depth” refers to the greatest depth (D) across the width (W) or length (L) of the recess. In one embodiment, the maximum depth (D) of the recess 710 is less than the length (L) of the recess 710. In one embodiment, the length (L) of the shallow recess 710 is at least 2, 3, 5, 10, 20, 50, 100, 200, 500, 700, 800, 900 or 1000 times greater than the depth (D) of the recess 710. In one embodiment, the maximum depth (D) of the recess 710 is greater than the width (W) of the recess 710. In another embodiment, the maximum depth (D) of the recess 710 is less than the width (W) of the recess 710. In another embodiment, the depth (D) of the recess 710 is less than both the width (W) and the length (L) of the recess. In one embodiment, the shallow recess 710 has an elongate shape in which the length (L) is at least 2, 5, 10, 50, 100 times greater than the width (W).

The depth (D), width (W) and length (L) of the shallow recess can be varied to vary the dosage of bioactive agent. Additionally, the dimensions of the shallow recess can vary depending upon the size of the implant. For example, the dimensions of a recess on a dental implant stem may be different than the dimensions of a recess on the stem of a prosthetic hip joint. In one embodiment, the dimensions of the recess are “shallow” relative to the overall dimensions of the device. For example, the recess on the stem may be less than 10%, less than 5%, less than 1% or less than 0.5% of the diameter or thickness of the stem. For example, a prosthetic hip joint typically has a diameter that is between about 6 mm and about 9 mm, or between about 7 mm and 8 mm. In one embodiment, the maximum depth of a shallow recess on a stem of a prosthetic hip joint may be less than 800 micrometers, less than 400 micrometers, less than 80 micrometers, or less than 40 micrometers. In other embodiment, the shallow recess has a maximum depth (D) that is less than about 500 micrometers, less than about 250 micrometers, less than about 100 micrometers or less than about 50 micrometers. In another embodiment, the shallow recess has a length that is at least about 1 millimeter, at least about 5 millimeters, at least about 10 millimeters, or at least about 100 millimeters. If desired, the surface 770 of the recess 710 can include one or more surface configurations to improve adhesion of the polymeric matrix. In one embodiment, one or more surface configurations include, for example, dimples, pockets, pores, raised portions (such as ridges or grooves), indented portions, and combinations thereof. In another embodiment, a surface configuration can be obtained by roughening the surface of the material, for example, using mechanical techniques (for example, using material such as 50 μm silica), chemical techniques, etching techniques, or other known methods. In another embodiment, a surface configuration can be obtained by using a porous material to fabricate at least a portion of the medical device. In another embodiment, the surface 770 of the recess 710 can be treated to provide pores. In still further embodiments, surface configuration can be accomplished by fabricating the device using a machined material, for example, machined metal.

Various configurations for the shallow recess are possible. Some non-limiting examples are shown in FIGS. 6A-6D. In the example shown in FIGS. 6A-6D, the implantable medical device 700 includes a stem 730 configured for insertion into a bone of a patient that has one or more shallow recesses 710 on a surface 720 of the stem 730. In one embodiment, shown in FIG. 6A, the stem has a length (SL) and a circumference and the recess 710 includes one or more elongate grooves that extend longitudinally along the length (SL) of the stem. In yet another embodiment, shown in FIG. 6B, the shallow recess 710 includes one or more elongate grooves that extend obliquely or in a spiral around the stem 730. In another embodiment, shown in FIG. 6C, the shallow recess 710 includes one or more elongate grooves that extend partially or completely around the circumference of the stem 730. In another embodiment, shown in FIG. 6D, the shallow recess 710 includes one or more recesses that form a pattern on the surface 720 of the stem 730. The term “pattern” refers to distinct, and often symmetrical geometric shapes and can include shapes such as lines, circles, ellipses, triangles, rectangles, and polygons, including, but not limited to, stripes, zigzags, crossed lines (perpendicular or oblique), grids, or a combination thereof. The shallow recess can also be formed in any combination of these or other configurations.

Another configuration for the shallow recess is shown in FIG. 7, in which the implantable medical device 700 includes threads 750 that have a surface 720 with one or more shallow recesses 710, in which a drug delivery matrix can be located. In another embodiment, the recesses may continue from the outer surface of the thread to the bottom of the thread as shown in FIG. 7. In one embodiment, the shallow recess 710 parallel to the axis AA-AA of the screw.

Drug Delivery Matrix

The invention can be used in connection with a wide variety of drug delivery compositions and matrices. As used herein, the term “drug delivery composition” refers to the vehicle that is placed within the shallow recess. The term “drug delivery matrix” refers to the composition once it is located within the shallow recess. The drug delivery composition can be provided in any suitable form, e.g., in the form of a true solution, or fluid or paste-like emulsion, mixture, dispersion or blend. A dried or cured drug delivery matrix can be obtained by removal of solvents or other volatile components and/or other physical-chemical actions (e.g., heating or illuminating) to solidify the coated composition within the recess.

Preferably, the drug delivery matrix is biocompatible, e.g., such that it results in no induction of inflammation or irritation when implanted. In one embodiment, the drug delivery composition includes a durable drug delivery matrix. As used herein the term “durable” refers to a matrix that is not intended to degrade during the useful life of the implant. In another embodiment, the drug delivery composition includes a biodegradable drug delivery matrix. In still other embodiments, the drug delivery composition includes a combination of both durable and biodegradable polymers.

In one embodiment, one or more bioactive agents are present in and can be released from the biodegradable matrix. In other embodiments one or more bioactive agents are present in a biodegradable microparticle, wherein the microparticle is immobilized within the polymeric matrix.

Durable Drug Delivery Systems

Many durable drug delivery systems are known and can be used in connection with the invention described herein. Suitable durable drug delivery systems are described in U.S. Pat. Nos. 7,008,667; 6,890,583, 6,344,035; 6,214,901; 6,890,583; 6,344,035; and 6,214,901, the disclosures of which are hereby incorporated by reference herein.

In one embodiment, the durable drug delivery composition includes a solvent, a combination of polymers dissolved in the solvent, and one or more bioactive agents dispersed in the polymer/solvent mixture. In one embodiment, the solvent is one in which the polymers are able to dissolve to form a solution. The bioactive agent may either be soluble in the solvent or form a dispersion throughout the solvent.

In one embodiment, the drug delivery composition includes a first polymer component that includes polyalkyl(meth)acrylate and/or aromatic poly(meth)acrylate. In a more particular embodiment, the poly(alkyl)(meth)acrylate polymers have alkyl chain lengths from 2 to 8 carbons, and with molecular weights from 50 kilodaltons to 900 kilodaltons. One example of a suitable poly(alkyl)(meth)acrylate is poly n-butylmethacrylate. In another embodiment, the first polymeric component includes polyaryl(meth)acrylates, polyaralkyl(meth)acrylates, polyaryloxyalkyl(meth)acrylates, or a combination thereof. The terms polyaryl(meth)acrylates, polyaralkyl(meth)acrylates, and polyaryloxyalkyl(meth)acrylates are used to describe polymeric structures wherein at least one carbon chain and at least one aromatic ring are combined with acrylic groups, typically esters. For example, a polyaralkyl(meth)acrylate or polyarylalky(meth)acrylate can be made from aromatic esters derived from alcohols also containing aromatic moieties. Examples of suitable first polymers include polyaryl(meth)acrylates, polyaralkyl(meth)acrylates, and polyaryloxyalkyl(meth)acrylates, in particular those with aryl groups having from 6 to 16 carbon atoms and with weight average molecular weights from about 50 to about 900 kilodaltons. Examples of polyaryl(meth)acrylates include poly-9-anthracenylmethacrylate, polychlorophenylacrylate, polymethacryloxy-2-hydroxybenzophenone, polymethacryloxybenzotriazole, polynaphthylacrylate, polynaphthylmethacrylate, poly-4-nitrophenylacrylate, polypentachloro(bromo, fluoro)acrylate and methacrylate, polyphenylacrylate and methacrylate. Examples of polyaralkyl(meth)acrylates include polybenzylacrylate and methacrylate, poly-2-phenethylacrylate and methacrylate, poly-1-pyrenylmethylmethacrylate. Examples of polyaryloxyalkyl(meth)acrylates include polyphenoxyethylacrylate and methacrylate, polyethyleneglycolphenylether acrylates and methacrylates with varying polyethyleneglycol molecular weights. The drug delivery composition also includes a second polymer component that includes poly(ethylene-co-vinyl acetate). In one embodiment, the poly(ethylene-co-vinyl acetate) polymer has vinyl acetate concentrations of between about 10% and about 50%.

Biodegradable Drug Delivery Systems

In one embodiment, the drug delivery composition is biodegradable. In a more particular embodiment, the biodegradable drug delivery composition includes a natural biodegradable polymer. In an alternate embodiment, the biodegradable drug delivery composition includes a synthetic biodegradable polymer.

Natural biodegradable polymers include polysaccharide and/or polysaccharide derivatives that are obtained from natural sources, such as plants or animals. Exemplary natural biodegradable polysaccharides include amylose, maltodextrin, amylopectin, starch, dextran, hyaluronic acid, heparin, chondroitin sulfate, dermatan sulfate, heparan sulfate, keratan sulfate, dextran sulfate, pentosan polysulfate, and chitosan. Preferred polysaccharides are low molecular weight polymers that have little or no branching, such as those that are derived from and/or found in starch preparations, for example, amylose and maltodextrin.

In another embodiment, the drug delivery composition includes one or more synthetic biodegradable polymers, such as polyanhydrides, polycaprolactone, polyglycolic acid, poly-L-lactic acid, poly-D-L-lactic, poly(D-lactic-co-glycolic acid), poly(D,L-lactic-co-glycolic acid), poly(ε-caprolactone), poly(lactic acid-co-lysine), poly(lactic acid-co-trimethylene carbonate), poly(valerolactone), poly(Hydroxyl butyrate), poly(hydrovalerate), polyphosphate esters, poly(hydroxybutyrate), polycarbonate, polyanhydride, poly(ortho esters), poly(phosphoesters), polyesters, polyamides, polyphosphazenes, poly(p-dioxane), poly(amino acid), polydioxanone, polypropylene fumarate), poly(ethyleneoxide), and poly(butyleneterephthalate).

Biodegradable Polymer

In one embodiment, the drug delivery composition can include one or more (e.g., 1, 2, 3 or 4) specific biodegradable polymers. Suitable polymers will be biodegradable and will be substantially soluble in the biocompatible solvent system. Specifically, the biodegradable polymer can have a solubility of at least about 50 g/L in the biocompatible solvent system, at 25° C. and 1 atm. In one embodiment, the biodegradable polymer will not include a polymer that is substantially insoluble in the biocompatible solvent system. In another embodiment, the biodegradable polymer will not include a biodegradable polymer that is substantially insoluble in water or bodily fluids.

Suitable specific classes of polymers include, e.g., polylactides, polyglycolides, polycaprolactones, polyanhydrides, polyamines, polyurethanes, polyesteramides, polyorthoesters, polydioxanones, polyacetals, polyketals, polycarbonates, polyorthocarbonates, polyphosphazenes, succinates, poly(malic acid), poly(amino acids), polyvinylpyrrolidone, polyethylene glycol, polyhydroxycellulose, polysaccharides, chitin, chitosan, and copolymers, block copolymers, multi-block co-polymers, multi-block co-polymers with polyethylene glycol (PEG), polyols, terpolymers and mixtures thereof.

In one embodiment, the biodegradable polymer is a thermoplastic polymer.

In one embodiment, the biodegradable polymer has a viscosity of at least about 100 cP at 37° C. In other embodiments, the biodegradable polymer has a viscosity of about 1,000 cP to about 30,000 cp at 37° C., about 5,000 cP to about 25,000 cp at 37° C., or about 10,000 cP to about 20,000 cp at 37° C.

In one embodiment, the biodegradable polymer is hydrophobic.

In one embodiment, the biodegradable polymer includes a block copolymer. In another embodiment, the biodegradable polymer is a polyethylene glycol (PEG) containing tri-block co-polymer.

In one embodiment the polymer contains functional side groups.

The biodegradable polymer can be present in any suitable and effective amount, provided the biodegradable polymer is substantially soluble in the solvent system, and in combination with the solvent system will form an implant in vivo. In one embodiment, the biodegradable polymer is present in about 10 wt. % to about 40 wt. % of the formulation. In another embodiment, the biodegradable polymer is present in about 40 wt. % to about 90 wt. % of the formulation.

In one embodiment, the biodegradable polymer can include a poly(ether ester) multi-block copolymer. In another embodiment, the biodegradable polymer can include a polyglycerol fatty acid ester. In another embodiment, the biodegradable polymer can include a PEG-PBT polymer. In another embodiment, the biodegradable polymer can include a CAMEO—polyester amide. In another embodiment, the biodegradable polymer can include a poly(ester-amide) polymer (PEA).

Poly(Ether Ester) Multi-Block Copolymers

One suitable class of biodegradable polymers useful in the present invention includes poly(ether ester) multi-block copolymers. These multi-block copolymers are composed of various pre-polymer building blocks of different combinations of DL-lactide, glycolide, ε-caprolactone and polyethylene glycol. By varying the molecular composition, molecular weight (Mw 1200-6000) and ratio of the pre-polymer blocks, different functionalities can be introduced into the final polymer, which enables the creation of polymers with various physio-chemical properties. Both hydrophobic as well as hydrophilic/swellable polymers and slowly degrading as well as rapidly degrading polymers can be designed.

The poly(ether ester) multi-block copolymers can include a polymer as shown below (formula III):

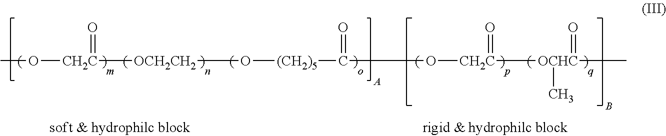

wherein,

m and p are each independently glycolide;

n is polyethylene glycol, Mw 300-1000;

o is ε-caprolactone; and

q is DL-lactide.

Under physiological conditions, poly(ether ester) multi-block copolymers can degrade completely via hydrolysis into non-toxic degradation products which are metabolized and/or excreted through the urinary pathway. Consequently, there can be no accumulation of biomaterials, thereby minimizing the chance of long-term foreign body reactions.

Additional features and descriptions of the poly(ether ester) multi-block copolymers are provided, for example, in Published PCT Patent Application No. WO 2005/068533 and references cited therein. An overview is provided below.

The multi-block a polymers can specifically include two hydiolysable segments having a different composition, linked by a multifunctional, specifically an aliphatic chain-extender, and which are specifically essentially completely amorphous under physiological conditions (moist environment, body temperature, which is approximately 37° C. for humans).

The resulting multi-block copolymers can specifically have a structure according to any of the formulae (1)-(3):

[—R₁—Q1—R₄—Q2—]ₓ—[R₂—Q3—R₄—Q4—]_{y}—[R₃—Q5—R₄—Q6—]_{z}— (1)

[—R₁—R₂—R₁—Q1—R₄—Q2—]ₓ—[R₃—Q2—R₄—Q1]_{z}— (2)

[—R₂—R₁—R₂—Q1—R₄—Q2—]ₓ—[R₃—Q2—R₄—Q1]_{z}— (3)

wherein

R₁ and R₂ can be amorphous polyester, amorphous poly ether ester or amorphous polycarbonate; or an amorphous pre-polymer that is obtained from combined ester, ether and/or carbonate groups. R₁ and R₂ can contain polyether groups, which can result from the use of these compounds as a polymerization initiator, the polyether being amorphous or crystalline at room temperature. However, the polyether thus introduced will become amorphous at physiological conditions. R₁ and R₂ are derived from amorphous pre-polymers or blocks A and B, respectively, and R₁ and R₂ are not the same. R₁ and R₂ can contain a polyether group at the same time. In a specific embodiment, only one of them will contain a polyether group;

z is zero or a positive integer;

R₃ is a polyether, such as poly(ethylene glycol), and may be present (z≠0) or not (z=0). Rx will become amorphous under physiological conditions;

R₄ is an aliphatic C₂-C₈ alkylene group, optionally substituted by a C₁-C₁₀ alkylene, the aliphatic group being linear or cyclic, wherein R₄ can specifically be a butylene, —(CH₂)₄— group, and the C₁-C₁₀ alkylene side group can contain protected S, N, P or O moieties;

x and y are both positive integers, which can both specifically be at least 1, whereas the sum of x and y (x+y) can specifically be at most 1000, more specifically at most 500, or at most 100. Q1-Q6 are linking units obtained by the reaction of the pre-polymers with the multifunctional chain-extender. Q1-Q6 are independently amine, urethane, amide, carbonate, ester or anhydride. The event that all linking groups Q are different being rare and not preferred.

Typically, one type of chain-extender can be used with three pre-polymers having the same end-groups, resulting in a copolymer of formula (1) with six similar linking groups. In case pre-polymers R₁ and R₂ are differently terminated, two types of groups Q will be present: e.g,. Q1 and Q2 will be the same between two linked pre-polymer segments R₁, but Q1 and Q2 are different when R₁ and R₂ are linked. Obviously, when Q1 and Q2 are the same, it means that they are the same type of group but as mirror images of each other.

In copolymers of formula (2) and (3) the groups Q1 and Q2 are the same when two pre-polymers are present that are both terminated with the same end-group (which is usually hydroxyl) but are different when the pre-polymers are differently terminated (e.g., PEG which is diol terminated and a di-acid terminated ‘tri-block’ pre-polymer). In case of the tri-block pre-polymers (R₁R₂R₁ and R₂R₁R₂), the outer segments should be essentially free of PEG, because the coupling reaction by ring opening can otherwise not be carried out successfully. Only the inner block can be initiated by a PEG molecule.

The examples of formula (1), (2) and (3) show the result of the reaction with a di-functional chain-extender and di-functional pre-polymers.

With reference to formula (1) the polyesters can also be represented as multi-block or segmented copolymers haying a structure (ab)n with alternating a and b segments or a structure (ab)r with a random distribution of segments a and b, wherein ‘a’ corresponds to the segment R₁ derived from pre-polymer (A) and ‘b’ corresponds to the segment R₂ derived from pre-polymer (B) (for z=0), In (ab)r, the a/b ratio (corresponding to x/y in formula (1)) may be unity or away from unity. The pre-polymers can be mixed in any desired amount and can be coupled by a multifunctional chain extender, viz, a compound having at least two functional groups by which it can be used to chemically link the pre-polymers. Specifically, this is a di-functional chain-extender. In case z≠0, then the presentation of a random distribution of all the segments can be given by (abc)r were three different pre-polymers (one being e.g. a polyethylene glycol) are randomly distributed in all possible ratio's. The alternating distribution is given by (abc)n. In this particular case, alternating means that two equally terminated pre-polymers (either a and c or b and c) are alternated with a differently terminated pre-polymer b or a, respectively, in an equivalent amount (a+c=b or b+c=a) Those according to formula (2) or (3) have a structure (aba)n and (bab)n. wherein the aba and bah ‘triblock’ pre-polymers are chain-extended with a di-functional molecule.

The method to obtain a copolymer with a random distribution of a and b (and optionally c) is far more advantageous than when the segments are alternating in the copolymer such as in (ab)n with the ratio of pre-polymers a and b being 1. The composition of the copolymer can then only be determined by adjusting the pre-polymer lengths. In general, the a and b segment lengths in (ab)n alternating copolymers are smaller than blocks in block-copolymers with structures ABA or AB.

The pre-polymers of which the a and b (and optionally c) segments are formed in (ab)r, (abc)r, (ab)n and (abc)n are linked by the di-functional chain-extender. This chain-extender can specifically he a diisocyanate chain-extender, but can also be a diacid or diol compound. In case all pre-polymers contain hydroxyl end-groups, the linking units will be urethane groups. In case (one of) the pre-polymers are carboxylic acid terminated, the linking units are amide groups. Multi-block copolymers with structure (ab)r and (abc)r can also be prepared by reaction of di-carboxylic acid terminated pre-polymers with a diol chain extender or vice versa (diol terminated pre-polymer with diacid chain-extender) using a coupling agent such as DCC (dicyclohexyl carbodiimide) forming ester linkages. In (aba)n and (bab)n the aba and bab pre-polymers are also specifically linked by an aliphatic di-functional chain-extender, more specifically, a diisocyanate chain-extender.

The term “randomly segmented” copolymers refers to copolymers that have a random distribution (i.e. not alternating) of the segments a and b: (ab)r or a, b and c: (abc)r.

PEG-PBT Polymers

One suitable class of biodegradable polymers useful in the present invention include poly(ether ester) multiblock copolymers based on poly(ethylene glycol) (PEG) and poly(butylene terephthalate) (PBT), that can be described by the following general formula IV:

[—(OCH₂CH₂)ₙ—O—C(O)—C₆H₄—C(O)—]ₓ[—O—(CH₂)₄—O—C(O)—C₆H₄—C(O)—]_{y} (IV)

wherein,

—C₆H₄— designates the divalent aromatic ring residue from each esterified molecule of terephthalic acid,

n represents the number of ethylene oxide units in each hydrophilic PEG block,

x represents the number of hydrophilic blocks in the copolymer, and

y represents the number of hydrophobic blocks in the copolymer.

In specific embodiments, n can be selected such that the molecular weight of the PEG block is between about 300 and about 4000. In specific embodiments, x and y can each be independently selected so that the multiblock copolymer contains from about 55% up to about 80% PEG by weight.

The block copolymer can be engineered to provide a wide array of physical characteristics (e.g., hydrophilicity, adherence, strength, malleability, degradability, durability, flexibility) and bioactive agent release characteristics (e.g., through controlled polymer degradation and swelling) by varying the values of n, x and y in the copolymer structure.

Polyester Amides

One suitable class of biodegradable polymers useful in the present invention includes the polyesteramide polymers having a subunit of the formula (V):

—[—O—(CH₂)ₓ—O—C(O)—CHR—NH—C(O)—(CH₂)_{y}—C(O)—NH—CHR—C(O)—]— (V)

wherein,

x is C₂-C₁₂,

y is C₂-C₁₂, and

R is —CH(CH₃₎₂, —CH₂CH(CH₃)₂, —CH(CH₃)CH₂CH₃, —CH₂(CH₂)₂CH₃, —CH₂C₆H₅,

—CH₂(CH₂)₂SCH₃ or part of an amino acid.

In specific embodiments, the C₂-C₁₂ can be (C₂-C₁₂) alkyl. In other specific embodiments, the C₂-C₁₂ can be (C₂-C₁₂) alkyl, optionally substituted.

Such polymers are described, for example, in U.S. Pat. No. 6,703,040. Polymers of this nature can be described with a nomenclature of x-aa-y, wherein “x” represents an alkyl diol with x carbon atoms, “aa” represents an amino acid such as leucine or phenylalanine, and y represents an alkyldicarboxylic acid with y carbon atoms, and wherein the polymer is a polymerization of the diol, the dicarboxylic acid, and the amino acid. An exemplary polymer of this type is 4-Leu-4.

Poly(Ester-Amide) Polymer (PEA)

One suitable class of biodegradable polymers useful in the present invention includes the poly(ester-amide) polymers. Such polymers can be prepared by polymerization of a diol, a dicarboxylic acid and an alpha-amino acid through ester and amide links in the form (DACA). An example of a (DACA)ₙ polymer is shown below in formula VI. Suitable amino acids include any natural or synthetic alpha-amino acid, specifically neutral amino acids.

Diols can be any aliphatic diol, including alkylene diols like HO—(CH₂)ₖ—OH (i.e. non-branched), branched diols (e.g., propylene glycol), cyclic diols (e.g. dianhydrohexitols and cyclohexanediol), or oligomeric diols based on ethylene glycol (e.g., diethylene glycol, triethylene glycol, tetraethylene glycol, or poly(ethylene glycol)s). Aromatic diols (e.g. bis-phenols) are less useful for these purposes since they are more toxic, and polymers based on them have rigid chains that are less likely to biodegrade.

Dicarboxylic acids can be any aliphatic dicarboxylic acid, such as α-omega-dicarboxylic acids (i.e., non-branched), branched dicarboxylic acids, cyclic dicarboxylic acids (e.g. cyclohexanedicarboxylic acid). Aromatic diacids (like phthalic acids, etc.) are less useful for these purposes since they are more toxic, and polymers based on them have rigid chain structure, exhibit poorer film-forming properties and have much lower tendency to biodegrade.

Specific PEA polymers have the formula VI:

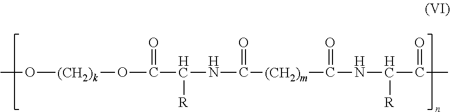

wherein,

k is 2-12 (e.g., 2, 3, 4, or 6);

m is 2-12 (e.g., 4 or 8); and

R is —CH(CH₃)₂, —CH₂CH(CH₃)₂, —CH(CH₃)CH₂CH₃, —CH₂(CH₂)₂CH₃, —CH₂(C₆H₅), or

### —CH₂(CH₂)SCH₃.

In specific embodiments, A is L-phenylalanine (Phe-PEA) and A is L-leucine (Leu-PEA). In specific embodiments, the ratio of Phe-PEA to Leu-PEA is from 10:1 to 1:1. In other specific embodiments, the ratio of Phe-PEA to Leu-PEA is from 5:1 to 2.5:1.

Additional features and descriptions of the poly(ester-amide) polymers (PEA) are provided, for example, in U.S. Re40,359, which is a reissue of U.S. Pat. No. 6,703,040.

Hydrophobic Derivatives of Natural Degradable Polysaccharides

One suitable class of biodegradable polymers useful in the present invention includes the hydrophobic derivatives of natural biodegradable polysaccharides. Hydrophobic derivatives of natural biodegradable polysaccharide refer to a natural biodegradable polysaccharide having one or more hydrophobic pendent groups attached to the polysaccharide. In many cases the hydrophobic derivative includes a plurality of groups that include hydrocarbon segments attached to the polysaccharide. When a plurality of groups including hydrocarbon segments are attached, they are collectively referred to as the “hydrophobic portion” of the hydrophobic derivative. The hydrophobic derivatives therefore include a hydrophobic portion and a polysaccharide portion.

The polysaccharide portion includes a natural biodegradable polysaccharide, which refers to a non-synthetic polysaccharide that is capable of being enzymatically degraded. Natural biodegradable polysaccharides include polysaccharide and/or polysaccharide derivatives that are obtained from natural sources, such as plants or animals. Natural biodegradable polysaccharides include any polysaccharide that has been processed or modified from a natural biodegradable polysaccharide (for example, maltodextrin is a natural biodegradable polysaccharide that is processed from starch). Exemplary natural biodegradable polysaccharides include maltodextrin, amylose, cyclodextrin, polyalditol, hyaluronic acid, dextran, heparin, chondroitin sulfate, dermatan sulfate, heparan sulfate, keratan sulfate, dextran, dextran sulfate, pentosan polysulfate, and chitosan. Specific polysaccharides are low molecular weight polymers that have little or no branching, such as those that are derived from and/or found in starch preparations, for example, maltodextrin, amylose, and cyclodextrin. Therefore, the natural biodegradable polysaccharide can be a substantially non-branched or completely non-branched poly(glucopyranose) polymer.

“Amylose” or “amylose polymer” refers to a linear polymer having repeating glucopyranose units that are joined by α-1,4 linkages. Some amylose polymers can have a very small amount of branching via α-1,6 linkages (about less than 0.5% of the linkages) but still demonstrate the same physical properties as linear (unbranched) amylose polymers do. Generally amylose polymers derived from plant sources have molecular weights of about 1×10⁶ Da or less. Amylopectin, comparatively, is a branched polymer having repeating glucopyranose units that are joined by α-1,4 linkages to form linear portions and the linear portions are linked together via α-1,6 linkages. The branch point linkages are generally greater than 1% of the total linkages and typically 4%-5% of the total linkages. Generally amylopectin derived from plant sources have molecular weights of 1×10⁷ Da or greater.

For example, in some aspects, starch preparations having a high amylose content, purified amylose, synthetically prepared amylose, or enriched amylose preparations can be used in the preparation of a hydrophobic derivative of amylose. In starch sources, amylose is typically present along with amylopectin, which is a branched polysaccharide. If a mixture of amylose and a higher molecular weight precursor is used (such as amylopectin), amylose can be present in the composition in an amount greater than the higher molecular weight precursor. For example, in some aspects, starch preparations having high amylose content, purified amylose, synthetically prepared amylose, or enriched amylose preparations can be used in the preparation of a hydrophobic derivative of amylose polymer. In some embodiments the composition includes a mixture of polysaccharides including amylose wherein the amylose content in the mixture of polysaccharides is 50% or greater, 60% or greater, 70% or greater, 80% or greater, or 85% or greater by weight. In other embodiments the composition includes a mixture of polysaccharides including amylose and amylopectin and wherein the amylopectin content in the mixture of polysaccharides is 30% or less, or 15% or less.

The amount of amylopectin present in a starch may also be reduced by treating the starch with amylopectinase, which cleaves α-1,6 linkages resulting in the debranching of amylopectin into amylose.

Steps may be performed before, during, and/or after the process of derivatizing the amylose polymer with a pendent group comprising a hydrocarbon segment to enrich the amount of amylose, or purify the amylose.

Amylose of particular molecular weights can be obtained commercially or can be prepared. For example, synthetic amyloses with average molecular masses of 70 kDa, 110 kDa, and 320 kDa, can be obtained from Nakano Vinegar Co., Ltd. (Aichi, Japan). The decision of using amylose of a particular size range may depend on factors such as the physical characteristics of the composition (e.g., viscosity), the desired rate of degradation of the implant, and the nature and amount of the active pharmaceutical ingredient (API).

Purified or enriched amylose preparations can be obtained commercially or can be prepared using standard biochemical techniques such as chromatography. In some aspects, high-amylose cornstarch can be used to prepare the hydrophobic derivative.

Maltodextrin is typically generated by hydrolyzing a starch slurry with heat-stable α-amylase at temperatures at 85-90° C. until the desired degree of hydrolysis is reached and then inactivating the α-amylase by a second heat treatment. The maltodextrin can be purified by filtration and then spray dried to a final product. Maltodextrins are typically characterized by their dextrose equivalent (DE) value, which is related to the degree of hydrolysis defined as: DE=MW dextrose/number-averaged MW starch hydrolysate X 100. Generally, maltodextrins are considered to have molecular weights that are less than amylose molecules.

A starch preparation that has been totally hydrolyzed to dextrose (glucose) has a DE of 100, whereas starch has a DE of about zero. A DE of greater than 0 but less than 100 characterizes the mean-average molecular weight of a starch hydrolysate, and maltodextrins are considered to have a DE of less than 20. Maltodextrins of various molecular weights, for example, in the range of about 500 Da to 5000 Da are commercially available (for example, from CarboMer, San Diego, Calif.).

Another contemplated class of natural biodegradable polysaccharides is natural biodegradable non-reducing polysaccharides. A non-reducing polysaccharide can provide an inert matrix thereby improving the stability of active pharmaceutical ingredients (APIs), such as proteins and enzymes. A non-reducing polysaccharide refers to a polymer of non-reducing disaccharides (two monosaccharides linked through their anomeric centers) such as trehalose (α-D-glucopyranosyl α-D-glucopyranoside) and sucrose (β-D-fructofuranosyl α-D-glucopyranoside). An exemplary non-reducing polysaccharide includes polyalditol which is available from GPC (Muscatine, Iowa). In another aspect, the polysaccharide is a glucopyranosyl polymer, such as a polymer that includes repeating (1→3)O-β-D-glucopyranosyl units.

Dextran is an α-D-1,6-glucose-linked glucan with side-chains 1-3 linked to the backbone units of the dextran biopolymer. Dextran includes hydroxyl groups at the 2, 3, and 4 positions on the glucopyranose monomeric units. Dextran can be obtained from fermentation of sucrose-containing media by Leuconostoc mesenteroides B512F.

Dextran can be obtained in low molecular weight preparations. Enzymes (dextranases) from molds such as Penicillium and Verticillium have been shown to degrade dextran. Similarly many bacteria produce extracellular dextranases that split dextran into low molecular weight sugars.

Chondroitin sulfate includes the repeating disaccharide units of D-galactosamine and D-glucuronic acid, and typically contains between 15 to 150 of these repeating units. Chondroitinase AC cleaves chondroitin sulfates A and C, and chondroitin.

Hyaluronic acid (HA) is a naturally derived linear polymer that includes alternating β-1,4-glucuronic acid and β-1,3-N-acetyl-D-glucosamine units. HA is the principal glycosaminoglycan in connective tissue fluids. HA can be fragmented in the presence of hyaluronidase.

In many aspects the polysaccharide portion and the hydrophobic portion include the predominant portion of the hydrophobic derivative of the natural biodegradable polysaccharide. Based on a weight percentage, the polysaccharide portion can be about 25% wt of the hydrophobic derivative or greater, in the range of about 25% to about 75%, in the range of about 30% to about 70%, in the range of about 35% to about 65%, in the range of about 40% to about 60%, or in the range of about 45% to about 55%. Likewise, based on a weight percentage of the overall hydrophobic derivative, the hydrophobic portion can be about 25% wt of the hydrophobic derivative or greater, in the range of about 25% to about 75%, in the range of about 30% to about 70%, in the range of about 35% to about 65%, in the range of about 40% to about 60%, or in the range of about 45% to about 55%. In exemplary aspects, the hydrophobic derivative has approximately 50% of its weight attributable to the polysaccharide portion, and approximately 50% of its weight attributable to its hydrophobic portion.

The hydrophobic derivative has the properties of being insoluble in water. The term for insolubility is a standard term used in the art, and meaning 1 part solute per 10,000 parts or greater solvent. (see, for example, Remington: The Science and Practice of Pharmacy, 20th ed. (2000), Lippincott Williams & Wilkins, Baltimore Md.).

A hydrophobic derivative can be prepared by associating one or more hydrophobic compound(s) with a natural biodegradable polysaccharide polymer. Methods for preparing hydrophobic derivatives of natural biodegradable polysaccharides are described herein.

The hydrophobic derivatives of the natural biodegradable polysaccharides specifically have an average molecular weight of up to about 1,000,000 Da, up to about 300,000 Da or up to about 100,000 Da. Use of these molecular weight derivatives can provide implants with desirable physical and drug-releasing properties. In some aspects the hydrophobic derivatives have a molecular weight of about 250,000 Da or less, about 100,000 Da or less, about 50,000 Da or less, or 25,000 Da or less. Particularly specific size ranges for the natural biodegradable polysaccharides are in the range of about 2,000 Da to about 20,000 Da, or about 4,000 Da to about 10,000 Da.

The molecular weight of the polymer is more precisely defined as “weight average molecular weight” or M_{w}. M_{w} is an absolute method of measuring molecular weight and is particularly useful for measuring the molecular weight of a polymer (preparation). Polymer preparations typically include polymers that individually have minor variations in molecular weight. Polymers are molecules that have a relatively high molecular weight and such minor variations within the polymer preparation do not affect the overall properties of the polymer preparation. The M_{w} can be measured using common techniques, such as light scattering or ultracentrifilgation. Discussion of M_{w} and other terms used to define the molecular weight of polymer preparations can be found in, for example, Allcock, H. R. and Lampe, F. W. (1990) Contemporary Polymer Chemistry; pg 271.

The addition of hydrophobic portion will generally cause an increase in molecular weight of the polysaccharide from its underivitized, starting molecular weight. The amount increase in molecular weight can depend on one or more factors, including the type of polysaccharide derivatized, the level of derivation, and, for example, the type or types of groups attached to the polysaccharide to provide the hydrophobic portion.

In some aspects, the addition of hydrophobic portion causes an increase in molecular weight of the polysaccharide of about 20% or greater, about 50% or greater, about 75% or greater, about 100% or greater, or about 125%, the increase in relation to the underivitized form of the polysaccharide.

As an example, a maltodextrin having a starting weight of about 3000 Da is derivitized to provide pendent hexanoate groups that are coupled to the polysaccharide via ester linkages to provide a degree of substitution (DS) of about 2.5. This provides a hydrophobic polysaccharide having a theoretical molecular weight of about 8400 Da.

In forming the hydrophobic derivative of the natural biodegradable polysaccharide and as an example, a compound having a hydrocarbon segment can be covalently coupled to one or more portions of the polysaccharide. For example, the compound can be coupled to monomeric units along the length of the polysaccharide. This provides a polysaccharide derivative with one or more pendent groups. Each chemical group includes a hydrocarbon segment. The hydrocarbon segment can constitute all of the pendent chemical group, or the hydrocarbon segment can constitute a portion of the pendent chemical group. For example, a portion of the hydrophobic polysaccharide can have the following structural formula (I):

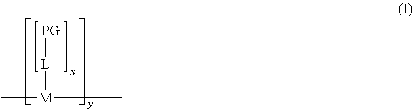

wherein each M is independently a monosaccharide unit, each L is independently a suitable linking group, or is a direct bond, each PG is independently a pendent group, each x is independently 0 to about 3, such that when x is 0, the bond between L and M is absent, and y is 3 or more.

Additionally, the polysaccharide that includes the unit of formula (I) above can be a compound of formula (II):

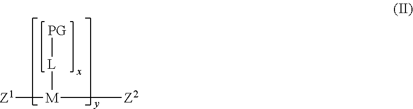

wherein each M is independently a monosaccharide unit, each L is independently a suitable linking group, or is a direct bond, each PG is independently a pendent group, each x is independently 0 to about 3, such that when x is 0, the bond between L and M is absent, y is about 3 to about 5,000, and Z¹ and Z² are each independently hydrogen, OR¹, OC(═O)R¹, CH₂OR¹, SiR¹ or CH₂OC(═O)R¹. Each R¹ is independently hydrogen, alkyl, cycloalkyl, cycloalkyl alkyl, aryl, aryl alkyl, heterocyclyl or heteroaryl, each alkyl, cycloalkyl, aryl, heterocycle and heteroaryl is optionally substituted, and each alkyl, cycloalkyl and heterocycle is optionally partially unsaturated.

For the compounds of formula (I) and (II), the monosaccharide unit (M) can include D-glucopyranose (e.g., α-D-glucopyranose). Additionally, the monosaccharide unit (M) can include non-macrocyclic poly-α(1→4) glucopyranose, non-macrocyclic poly-α(1→6) glucopyranose, or a mixture or combination of both non-macrocyclic poly-α(1→4) glucopyranose and non-macrocyclic poly-α(1→6) glucopyranose. For example, the monosaccharide unit (M) can include glucopyranose units, wherein at least about 90% are linked by α(1→4) glycosidic bonds. Alternatively, the monosaccharide unit (M) can include glucopyranose units, wherein at least about 90% are linked by α(1→6) glycosidic bonds. Additionally, each of the monosaccharides in the polysaccharide can be the same type (homopolysaccharide), or the monosaccharides in the polysaccharide can differ (heteropolysaccharide).

The polysaccharide can include up to about 5,000 monosaccharide units (i.e., y in the formula (I) or (II) is up to 5,000). Specifically, the monosaccharide units can be glucopyranose units (e.g., α-D-glucopyranose units). Additionally, y in the formula (I) or (II) can specifically be about 3-5,000 or about 3-4,000 or about 100 to 4,000.

In specific embodiments, the polysaccharide is non-macrocyclic. In other specific embodiments, the polysaccharide is linear. In other specific embodiments, the polysaccharide is branched. In yet further specific embodiments, the polysaccharide is a natural polysaccharide (PS).

The polysaccharide will have a suitable glass transition temperature (Tg). In one embodiment, the polysaccharide will have a glass transition temperature (Tg) of at least about 35° C. (e.g., about 40° C. to about 150° C.). In another embodiment, the polysaccharide will have a glass transition temperature (Tg) of −30° C. to about 0° C.

A “pendant group” refers to a group of covalently bonded carbon atoms having the formula (CHₙ)ₘ, wherein m is 2 or greater, and n is independently 2 or 1. A hydrocarbon segment can include saturated hydrocarbon groups or unsaturated hydrocarbon groups, and examples thereof include alkyl, alkenyl, alkynyl, cyclic alkyl, cyclic alkenyl, aromatic hydrocarbon and aralkyl groups. Specifically, the pendant group includes linear, straight chain or branched C₁-C₂₀ alkyl group; an amine terminated hydrocarbon or a hydroxyl terminated hydrocarbon. In another embodiment, the pendant group includes polyesters such as polylactides, polyglycolides, poly (lactide-co-glycolide) co-polymers, polycaprolactone, terpolymers of poly (lactide-co-glycolide-co-caprolatone), or combinations thereof.

The monomeric units of the hydrophobic polysaccharides described herein typically include monomeric units having ring structures with one or more reactive groups. These reactive groups are exemplified by hydroxyl groups, such as the ones that are present on glucopyranose-based monomeric units, e.g., of amylose and maltodextrin. These hydroxyl groups can be reacted with a compound that includes a hydrocarbon segment and a group that is reactive with the hydroxyl group (a hydroxyl-reactive group).

Examples of hydroxyl reactive groups include acetal, carboxyl, anhydride, acid halide, and the like. These groups can be used to form a hydrolytically cleavable covalent bond between the hydrocarbon segment and the polysaccharide backbone. For example, the method can provide a pendent group having a hydrocarbon segment, the pendent group linked to the polysaccharide backbone with a cleavable ester bond. In these aspects, the synthesized hydrophobic derivative of the natural biodegradable polysaccharide can include chemical linkages that are both enzymatically cleavable (the polymer backbone) and non-enzymatically hydrolytically cleavable (the linkage between the pendent group and the polymer backbone).

Other cleavable chemical linkages (e.g., metabolically cleavable covalent bonds) that can be used to bond the pendent groups to the polysaccharide include carboxylic ester, carbonate, borate, silyl ether, peroxyester groups, disulfide groups, and hydrazone groups.

In some cases, the hydroxyl reactive groups include those such as isocyanate and epoxy. These groups can be used to form a non-cleavable covalent bond between the pendent group and the polysaccharide backbone. In these aspects, the synthesized hydrophobic derivative of the natural biodegradable polysaccharide includes chemical linkages that are enzymatically cleavable.

Other reactive groups, such as carboxyl groups, acetyl groups, or sulphate groups, are present on the ring structure of monomeric units of other natural biodegradable polysaccharides, such as chondrotin or hyaluronic acid. These groups can also be targeted for reaction with a compound having a hydrocarbon segment to be bonded to the polysaccharide backbone.

Various factors can be taken into consideration in the synthesis of the hydrophobic derivative of the natural biodegradable polysaccharide. These factors include the physical and chemical properties of the natural biodegradable polysaccharide, including its size, and the number and presence of reactive groups on the polysaccharide and solubility, the physical and chemical properties of the compound that includes the hydrocarbon segment, including its the size and solubility, and the reactivity of the compound with the polysaccharide.

In preparing the hydrophobic derivative of the natural biodegradable polysaccharide any suitable synthesis procedure can be performed. Synthesis can be carried out to provide a desired number of groups with hydrocarbon segments pendent from the polysaccharide backbone. The number and/or density of the pendent groups can be controlled, for example, by controlling the relative concentration of the compound that includes the hydrocarbon segment to the available reactive groups (e.g., hydroxyl groups) on the polysaccharide.

The type and amount of groups having the hydrocarbon segment pendent from the polysaccharide is sufficient for the hydrophobic polysaccharide to be insoluble in water. In order to achieve this, as a general approach, a hydrophobic polysaccharide is obtained or prepared wherein the groups having the hydrocarbon segment pendent from the polysaccharide backbone in an amount in the range of 0.25 (pendent group): 1 (polysaccharide monomer) by weight.

The weight ratio of glucopyranose units to pendent groups can vary, but will typically be about 1:1 to about 100:1. Specifically, the weight ratio of glucopyranose units to pendent groups can be about 1:1 to about 75:1, or about 1:1 to about 50:1. Additionally, the nature and amount of the pendent group can provide a suitable degree of substitution to the polysaccharide. Typically, the degree of substitution will be in the range of about 0.1-5 or about 0.5-2.

To exemplify these levels of derivation, very low molecular weight (less than 10,000 Da) glucopyranose polymers are reacted with compounds having the hydrocarbon segment to provide low molecular weight hydrophobic glucopyranose polymers. In one mode of practice, the natural biodegradable polysaccharide maltodextrin in an amount of 10 g (MW 3000-5000 Da; ˜3 mmols) is dissolved in a suitable solvent, such as tetrahydrofuran. Next, a solution having butyric anhydride in an amount of 18 g (0.11 mols) is added to the maltodextrin solution. The reaction is allowed to proceed, effectively forming pendent butyrate groups on the pyranose rings of the maltodextrin polymer. This level of derivation results in a degree of substitution (DS) of butyrate group of the hydroxyl groups on the maltodextrin of about 1.

For maltodextrin and other polysaccharides that include three hydroxyl groups per monomeric unit, on average, one of the three hydroxyl groups per glycopyranose monomeric unit becomes substituted with a butyrate group. A maltodextrin polymer having this level of substitution is referred to herein as maltodextrin-butyrate DS 1. As described herein, the DS refers to the average number of reactive groups (including hydroxyl and other reactive groups) per monomeric unit that are substituted with pendent groups comprising hydrocarbon segments.

An increase in the DS can be achieved by incrementally increasing the amount of compound that provides the hydrocarbon segment to the polysaccharide. As another example, butyrylated maltodextrin having a DS of 2.5 is prepared by reacting 10 g of maltodextrin (MW 3000-5000 Da; ˜3 mmols) with 0.32 mols butyric anhydride.

The degree of substitution can influence the hydrophobic character of the polysaccharide. In turn, implants formed from hydrophobic derivatives having a substantial amount of groups having the hydrocarbon segments bonded to the polysaccharide backbone (as exemplified by a high DS) are generally more hydrophobic and can be more resistant to degradation. For example, an implant formed from maltodextrin-butyrate DS1 has a rate of degradation that is faster than an implant formed from maltodextrin-butyrate DS2.

The type of hydrocarbon segment present in the groups pendent from the polysaccharide backbone can also influence the hydrophobic properties of the polymer. In one aspect, the implant is formed using a hydrophobic polysaccharide having pendent groups with hydrocarbon segments being short chain branched alkyl group. Exemplary short chain branched alkyl group are branched C₄-C₁₀ groups. The preparation of a hydrophobic polymer with these types of pendent groups is exemplified by the reaction of maltodextrin with valproic acid/anhydride with maltodextrin (MD-val). The reaction can be carried out to provide a relatively lower degree of substitution of the hydroxyl groups, such as is in the range of 0.5-1.5. Although these polysaccharides have a lower degree of substitution, the short chain branched alkyl group imparts considerable hydrophobic properties to the polysaccharide.

Even at these low degrees of substitution the MD-val forms coatings that are very compliant and durable. Because of the low degrees of substitution, the pendent groups with the branched C₈ segment can be hydrolyzed from the polysaccharide backbone at a relatively fast rate, thereby providing a biodegradable coatings that have a relatively fast rate of degradation.

For polysaccharides having hydrolytically cleavable pendent groups that include hydrocarbon segments, penetration by an aqueous solution can promote hydrolysis and loss of groups pendent from the polysaccharide backbone. This can alter the properties of the implant, and can result in greater access to enzymes that promote the degradation of the natural biodegradable polysaccharide.

Various synthetic schemes can be used for the preparation of a hydrophobic derivative of a natural biodegradable polysaccharide. In some modes of preparation, pendent polysaccharide hydroxyl groups are reacted with a compound that includes a hydrocarbon segment and a group that is reactive with the hydroxyl groups. This reaction can provide polysaccharide with pendent groups comprising hydrocarbon segments.

Any suitable chemical group can be coupled to the polysaccharide backbone and provide the polysaccharide with hydrophobic properties, wherein the polysaccharide becomes insoluble in water. Specifically, the pendent group can include one or more atoms selected from carbon (C), hydrogen (H), oxygen (O), nitrogen (N), and sulfur (S).

In some aspects, the pendent group includes a hydrocarbon segment that is a linear, branched, or cyclic C₂-C₁₈ group. More specifically the hydrocarbon segment includes a C₂-C₁₀, or a C₄-C₈, linear, branched, or cyclic group. The hydrocarbon segment can be saturated or unsaturated, and can include alkyl groups or aromatic groups, respectively. The hydrocarbon segment can be linked to the polysaccharide chain via a hydrolyzable bond or a non-hydrolyzable bond.

In some aspects the compound having a hydrocarbon segment that is reacted with the polysaccharide backbone is derived from a natural compound. Natural compounds with hydrocarbon segments include fatty acids, fats, oils, waxes, phospholipids, prostaglandins, thromboxanes, leukotrienes, terpenes, steroids, and lipid soluble vitamins.

Exemplary natural compounds with hydrocarbon segments include fatty acids and derivatives thereof, such as fatty acid anhydrides and fatty acid halides. Exemplary fatty acids and anhydrides include acetic, propionic, butyric, isobutyric, valeric, caproic, caprylic, capric, and lauric acids and anhydrides, respectively. The hydroxyl group of a polysaccharide can be reacted with a fatty acid or anhydride to bond the hydrocarbon segment of the compound to the polysaccharide via an ester group.

The hydroxyl group of a polysaccharide can also cause the ring opening of lactones to provide pendent open-chain hydroxy esters. Exemplary lactones that can be reacted with the polysaccharide include caprolactone and glycolides.

Generally, if compounds having large hydrocarbon segments are used for the synthesis of the hydrophobic derivative, a smaller amount of the compound may be needed for its synthesis. For example, as a general rule, if a compound having a hydrocarbon segments with an alkyl chain length of Cₓ is used to prepare a hydrophobic derivative with a DS of 1, a compound having a hydrocarbon segment with an alkyl chain length of C_{(x×2)} is reacted in an amount to provide a hydrophobic derivative with a DS of 0.5.

The hydrophobic derivative of the natural biodegradable polysaccharide can also be synthesized having combinations of pendent groups with two or more different hydrocarbon segments, respectively. For example, the hydrophobic derivative can be synthesized using compounds having hydrocarbon segments with different alkyl chain lengths. In one mode of practice, a polysaccharide is reacted with a mixture of two or more fatty acids (or derivatives thereof) selected from the group of acetic acid, propionic acid, butyric acid, isobutyric acid, valeric acid, caproic acid, caprylic acid, capric acid, and lauric acid to generate the hydrophobic derivative.

In other cases the hydrophobic derivative is synthesized having a non-hydrolyzable bond linking the hydrocarbon segment to the polysaccharide backbone. Exemplary non-hydrolyzable bonds include urethane bonds.

The hydrophobic derivative of the natural biodegradable polysaccharide can also be synthesized so that hydrocarbon segments are individually linked to the polysaccharide backbone via both hydrolyzable and non-hydrolyzable bonds. As another example, a hydrophobic derivative is prepared by reacting a mixture of butyric acid anhydride and butyl isocyanate with maltodextrin. This yields a hydrophobic derivative of maltodextrin with pendent butyric acid groups that are individually covalently bonded to the maltodextrin backbone with hydrolyzable ester linkages and non-hydrolyzable urethane linkages. The degradation of a coating having this type of hydrophobic derivative can occur by loss of the butyrate groups from hydrolysis of the ester linkages. However, a portion of the butyrate groups (the ones that are bonded via the urethane groups) are not removed from the polysaccharide backbone and therefore the natural biodegradable polysaccharide can maintain a desired degree of hydrophobicity, prior to enzymatic degradation of the polysaccharide backbone.

In some aspects, the group that is pendent from the polysaccharide backbone has properties of an active pharmaceutical ingredient (API). In this regard, the implants include polysaccharide-coupled API. In some aspects, an API which has a hydrocarbon segment can be hydrolyzed from the natural biodegradable polymer and released from the matrix to provide a therapeutic effect. One example of a therapeutically useful compound having a hydrocarbon segments is butyric acid, which has been shown to elicit tumor cell differentiation and apoptosis, and is thought to be useful for the treatment of cancer and other blood diseases.

Other illustrative compounds that include hydrocarbon segments include valproic acid and retinoic acid. These compounds can be coupled to a polysaccharide backbone to provide a pendent group, and then cleaved from the polysaccharide backbone upon degradation of the implant in vivo. Retinoic acid is known to possess antiproliferative effects and is thought to be useful for treatment of proliferative vitreoretinopathy (PVR). The pendent group that provides a therapeutic effect can also be a natural compound (such as butyric acid, valproic acid, and retinoic acid).

Another illustrative class of compounds that can be coupled to the polysaccharide backbone is the corticosteroids. An exemplary corticosteroid is triamcinolone. One method of coupling triamcinolone to a natural biodegradable polymer is by employing a modification of the method described in Cayanis, E. et al., Generation of an Auto-anti-idiotypic Antibody that Binds to Glucocorticoid Receptor, The Journal of Biol. Chem., 261(11): 5094-5103 (1986). Triamcinolone hexanoic acid is prepared by reaction of triamcinolone with ketohexanoic acid; an acid chloride of the resulting triamcinolone hexanoic acid can be formed and then reacted with the natural biodegradable polymer, such as maltodextrin or polyalditol, resulting in pendent triamcinolone groups coupled via ester bonds to the natural biodegradable polymer.

The hydrophobic derivative of the natural biodegradable polysaccharide can also be synthesized having two or more different pendent groups, wherein at least one of the pendent groups includes an API. The hydrophobic polysaccharide can be synthesized with an amount of a pendent groups including an API, that when released from the polysaccharide, provides a therapeutic effect to the subject. An example of such a hydrophobic derivative is maltodextrin-caproate-triamcinolone. This hydrophobic derivative can be prepared by reacting a mixture including triamcinolone hexanoic acid and an excess of caproic anhydride (n-hexanoic anhydride) with maltodextrin to provide a derivative with a DS of 2.5.

In some aspects, the group that is pendent from the polysaccharide includes a hydrocarbon segment that is an aromatic group, such as a phenyl group. As one example, o-acetylsalicylic acid is reacted with a polysaccharide such as maltodextrin to provide pendent chemical group having a hydrocarbon segment that is a phenyl group, and a non-hydrocarbon segment that is an acetate group wherein the pendent group is linked to the polysaccharide via an ester bond.

Additional features and descriptions of the biodegradable polymers that include the hydrophobic derivatives of natural biodegradable polysaccharides can be found, for example, in U.S. Patent Publication Nos. 2007/0218102, 2007/0260054 and 2007/0224247, and references cited therein.

Bioactive Agent

In one embodiment, the drug delivery composition includes one or more bioactive agents. The term “bioactive agent,” refers to an inorganic or organic molecule, which can be synthetic or natural, that causes a biological effect when administered in vivo to an animal, including but not limited to birds and mammals, including humans. The bioactive agents can be present in the matrix in amount to provide a desired therapeutic response during a period of time the device is implanted in a subject. The amount of bioactive agent placed within the delivery device may depend on one or more factors including: the potency of the bioactive agents, the in vivo lifetime of the bioactive agents, and the desired release rate of the drugs. While the invention is not limited to any particular amount of bioactive agents that are present within the device, amounts of bioactive agents present can be in very small amounts, such as in picogram amounts, up to very large quantities, such as gram amounts.

A partial list of bioactive agents is provided below. A comprehensive listing of bioactive agents, in addition to information of the water solubility of the bioactive agents, can be found in The Merck Index, Fourteenth Edition, Merck & Co. (2006).

The drug delivery matrix can be used to release bioactive agents falling within one or more classes that include, but are not limited to: ACE inhibitors, actin inhibitors, analgesics, anesthetics, anti-hypertensives, anti polymerases, antisecretory agents, anti-AIDS substances, antibiotics, anti-cancer substances, anti-cholinergics, anti-coagulants, anti-convulsants, anti-depressants, anti-emetics, antifungals, antimicrobials, anti-glaucoma solutes, antihistamines, antihypertensive agents, anti-inflammatory agents (such as NSAIDs), anti metabolites, antimitotics, antioxidizing agents, anti-parasite and/or anti-Parkinson substances, antiproliferatives (including antiangiogenesis agents), anti-protozoal solutes, anti-psychotic substances, anti-pyretics, antiseptics, anti-spasmodics, antiviral agents, calcium channel blockers, cell response modifiers, chelators, chemotherapeutic agents, dopamine agonists, extracellular matrix components, fibrinolytic agents, free radical scavengers, growth hormone antagonists, hypnotics, immunosuppressive agents, immunotoxins, inhibitors of surface glycoprotein receptors, microtubule inhibitors, miotics, muscle contractants, muscle relaxants, neurotoxins, neurotransmitters, polynucleotides and derivatives thereof, opioids, photodynamic therapy agents, prostaglandins, remodeling inhibitors, statins, steroids, thrombolytic agents, tranquilizers, vasodilators, and vasospasm inhibitors.

The term “antimicrobial agent” as used in the present invention means antibiotics, antiseptics, disinfectants and other synthetic moieties, and combinations thereof, that are soluble in organic solvents such as alcohols, ketones, ethers, aldehydes, acetonitrile, acetic acid, formic acid, methylene chloride and chloroform.

Classes of antibiotics include tetracyclines (i.e. minocycline), rifamycins (i.e. rifampin), macrolides (i.e. erythromycin), penicillins (i.e. nafcillin), cephalosporins (i.e. cefazolin), other beta-lactam antibiotics (i.e. imipenem, aztreonam), aminoglycosides (i.e. gentamicin), chloramphenicol, sufonamides (i.e. sulfamethoxazole), glycopeptides (i.e. vancomycin), quinolones (i.e. ciprofloxacin), fusidic acid, trimethoprim, metronidazole, clindamycin, mupirocin, polyenes (i.e. amphotericin B), azoles (i.e. fluconazole) and beta-lactam inhibitors (i.e. sulbactam). Additional examples of antibiotics that can be used include teicoplanin, azithromycin, clarithromycin, ofloxacin, lomefloxacin, norfloxacin, nalidixic acid, sparfloxacin, pefloxacin, amifloxacin, enoxacin, fleroxacin, temafloxacin, tosufloxacin, clinafloxacin, clavulanic acid, itraconazole, ketoconazole, and nystatin.

Antiseptics are recognized as substances that prevent or arrest the growth or action of microorganisms, generally in a nonspecific fashion, e.g., by inhibiting their activity or destroying them. Examples of antiseptics include silver sulfadiazine, peracetic acid, sodium hypochlorite, phenols, phenolic compounds, iodophor compounds, quaternary ammonium compounds, and chlorine compounds. Additional examples of antiseptics and disinfectants include thymol, a-terpineol, methylisothiazolone, cetylpyridinium, chloroxylenol, hexachlorophene, cationic biguanides (i.e. chlorhexidine, cyclohexidine), methylene chloride, iodine and iodophores (i.e. povidone-iodine), triclosan, furan medical preparations (i.e. nitrofurantoin, nitrofurazone), methenamine, aldehydes (glutaraldehyde, formaldehyde) and alcohols. Other examples of antiseptics and disinfectants will readily suggest themselves to those of ordinary skill in the art.

Anti-viral agents are substances capable of destroying or suppressing the replication of viruses. Examples of anti-viral agents include α-methyl-P-adamantane methylamine, hydroxy-ethoxymethylguanine, adamantanamine, 5-iodo-2′-deoxyuridine, trifluorothymidine, interferon, and adenine arabinoside.

Anti-pyretics are substances capable of relieving or reducing fever. Anti-inflammatory agents are substances capable of counteracting or suppressing inflammation. Examples of such agents include aspirin (salicylic acid), indomethacin, sodium indomethacin trihydrate, salicylamide, naproxen, colchicine, fenoprofen, sulindac, diflunisal, diclofenac, indoprofen and sodium salicylamide. Local anesthetics are substances that have an anesthetic effect in a localized region. Examples of such anesthetics include procaine, lidocaine, tetracaine and dibucaine.

Method of Making

One or more shallow recesses can be formed in an implantable medical device in any suitable manner using known technology. For example, after obtaining the desired device, shallow recesses can be formed using electrical discharge machining (EDM), laser engraving, multi-axis milling, or a combination thereof.

EDM refers to a machining method used for electrically conductive metals to cut small odd-shaped angles, detailed contours or cavities in hardened steel as well as other metals, including, but not limited to titanium. Wire EDM Machining (also known as Spark EDM), refers to an electro thermal process in which a thin single-strand metal wire (usually brass) is able to cut through metal using the heat from electrical sparks. Wire EDM can easily machine complex parts and precision components out of hard conductive materials. Sinker EDM Machining refers to a process in which two metal parts are submerged in an insulating liquid and connected to a source of current which is switched on and off automatically depending on the parameters set on the controller. When the current is switched on, an electric tension is created between the two metal parts. If the two parts are brought in close proximity, the electrical tension is discharged and a spark jumps across. Where it strikes, the metal is heated and melts.

Multiaxis milling refers to a manufacturing process in which computer numerical controlled (CNC) tools move to mill away excess material, for example, by water jet cutting or laser cutting. Whereas typical CNC tools support translation in three axes, multiaxis machines support rotation around one or multiple axes, typically between four and nine axes. Each axis of movement is implemented either by moving the table (into which the workpiece is attached), or by moving the tool. The actual configuration of axes varies, therefore machines with the same number of axes can differ in the movements that can be performed.

Once the shallow recesses are formed, a suitable drug delivery composition can be introduced into one or more shallow recesses. As discussed above, inclusion of a drug delivery matrix in a shallow recess on a surface of an implantable medical device can be beneficial, especially when the surface is exposed to frictional forces, for example, during implantation and/or use. Therefore, in one embodiment, the recess is “underfilled” with the drug delivery composition. For example, to protect the drug delivery matrix from shear or frictional forces, the volume of drug delivery matrix that is placed in the recess is less than the volume defined by the recess, wherein the volume of the recess is defined by the length (L), width (W) and depth (D) of the recess. In another embodiment, the recess has a maximum depth (D) and the drug delivery matrix has a thickness T that is less than the maximum depth (D) of the recess (See, FIG. 5).

In one embodiment, overfilling of the recess or overspray onto the adjacent surface is reduced or prevented by masking one or more frictional surfaces of the device before the drug delivery composition is introduced. In one embodiment, the drug delivery composition is introduced into the shallow recess from a solution in which the bioactive agent and matrix are dissolved and precipitate upon drying of the solvent. In another embodiment, the drug delivery composition is introduced into the shallow recess from suspension in which the matrix is dissolved and the bioactive agent is suspended in solid form, wherein the matrix precipitates upon drying of the solvent. In yet another embodiment, the drug delivery composition is formed as a molten mixture and the drug delivery composition is introduced through an insert molding or melt extrusion process. After deposition, the matrix material cools and solidifies. In the case of melt extrusion, a deposition nozzle may be moved in a controlled fashion over the shallow recess to fill the recess with the desired amount. Alternatively, the medical device may be moved in relation to a fixed extrusion nozzle. In one embodiment, both the polymeric matrix and bioactive agent is melted . In another embodiment, the polymeric matrix is melted, but the bioactive agent is not and, instead, is mixed within the polymeric matrix. In another embodiment, the drug delivery matrix is introduced using ultrasonic or aerosol jet spray technology. In one embodiment, columnated spray technology, such as the technology available from Optomec (Albuquerque, NM) can be used to reduce overspray of the drug delivery composition onto the adjacent surface of the device.

Because the drug delivery matrix is isolated from frictional forces, there is no need to include a protective covering over the top of the recess or drug delivery matrix. However, if desired, the drug delivery matrix can be formed as one or more layers. In one embodiment, the drug delivery matrix includes a topcoat. In contrast to a protective coating, which is configured to shield the matrix from frictional forces or a barrier, which is configured to help retain the matrix within the recess, a topcoat may be provided to control the rate at which the bioactive agent is released from the matrix. In one embodiment, the topcoat is formed using the same polymeric components as the drug delivery matrix, either with or without bioactive agent. In one embodiment, the drug delivery matrix within the recess has a maximum thickness that is up to 10 times greater, up to 25 times greater, up to 50 times greater, or up to 100 times greater than the average thickness of the topcoat. For example, the drug delivery matrix within the recess may have a thickness of less than about 500 micrometers and the topcoat may have a thickness of between about 5 micrometers and about 10 micrometers.

If desired, the drug delivery composition can be introduced under conditions of controlled relative humidity. Humidity can be “controlled” in any suitable manner, including at the time of preparing and/or using (as by applying) the composition (for instance, by applying the composition to the device in a confined chamber or area adapted to provide a relative humidity different than ambient conditions), and/or by adjusting the water content of the composition itself. In turn, even ambient humidity can be considered “controlled” humidity, if indeed it has been correlated with and determined to provide a corresponding controlled bioactive release profile.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The foregoing discloses embodiments of the invention. In the Specification and claims, the term “about” is used to modify, for example, the quantity of an ingredient in a composition, concentration, volume, process, and similar values and ranges thereof, to describe various embodiments of the disclosure. The term “about” refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods, and like proximate considerations. The term “about” also encompasses amounts that differ due to aging of a formulation with a particular initial concentration or mixture, and amounts that differ due to mixing or processing a formulation with a particular initial concentration or mixture. Where modified by the term “about” the claims appended hereto include equivalents to these quantities.

All publications and patent applications in this specification are indicative of the level of ordinary skill in the art to which this invention pertains. All publications and patent applications are herein incorporated by reference to the same extent as if each individual publication or patent application was specifically and individually indicated by reference.

This application is intended to cover adaptations or variations of the present subject matter. It is to be understood that the above description is intended to be illustrative, and not restrictive. It should be readily apparent that any one or more of the design features described herein may be used in any combination with any particular configuration. With use of the metal injection molding process, such design features can be incorporated without substantial additional manufacturing costs. That the number of combinations are too numerous to describe, and the present invention is not limited by or to any particular illustrative combination described herein. The scope of the present subject matter should be determined with reference to the appended claims, along with the full scope of equivalents to which such claims are entitled.

What is claimed is:

1. An implantable medical device comprising:
one or more surfaces that include one or more shallow recesses configured to receive a drug delivery composition comprising a polymeric matrix and one or more biologically active agents.

2. The implantable medical device of claim 1, wherein one or more surfaces comprises a frictional surface.

3. The implantable medical device of claim 1, wherein the implantable medical device comprises a stem configured for insertion into a bone of a patient, wherein the stem comprises one or more surfaces that include one or more shallow recesses configured to receive the drug delivery composition.

4. The implantable medical device of claim 1, wherein the shallow recess has a length and a depth and the depth is less than the length.

5. The implantable medical device of claim 2, wherein the length of the shallow recess is at least 2 times greater than the depth.

6. The implantable medical device of claim 2, wherein the shallow recess has a depth of less than about 500 micrometers and a length of at least about 1 millimeter.

7. The implantable medical device of claim 1, wherein the shallow recess defines a volume that is equal to or greater than a volume of drug delivery composition introduced into the recess.

8. The implantable medical device of claim 1, wherein the maximum depth of the shallow recess is greater than a thickness of drug delivery composition introduced into the recess.

9. The implantable medical device of claim 1, wherein the recess has a surface with one or more surface configurations to improve adhesion of the drug delivery composition.

10. The implantable medical device of claim 1, wherein the stem has a length and a circumference and the recess comprises an elongate groove selected from an elongate groove extending longitudinally along the length of the stem, an elongate groove extending partially or completely around the circumference of the stem; an elongate groove extending obliquely or in a spiral around the stem; a pattern of elongate grooves applied to the surface of the stem, or a combination thereof.

11. The implantable medical device of claim 1, wherein the number, size and distribution of recesses can be varied to vary dosage of the biologically active agent.

12. The implantable medical device of claim 1, wherein the recess has a cross sectional shape that is circular, parabolic, or polygonal, or a combination thereof.

13. The implantable medical device of claim 11, wherein the recess has a polygonal cross sectional shape selected from triangular, rectangular, square, trapezoidal, or a combination thereof.

14. The implantable medical device of claim 1, wherein the recess has a cross sectional size that is selected from a cross sectional size that is constant along the length of the recess, a cross sectional size that varies along the length of the recess, and combinations thereof.

15. The implantable medical device of claim 1, wherein the biologically active agent is selected from an agent that is antimicrobial, antibiotic, anti-inflammatory agent, anticoagulant, growth factor, or other therapeutic agent.

16. The implantable medical device of claim 1, wherein the drug delivery composition comprises a durable drug delivery composition, a biodegradable drug delivery composition, or a combination thereof.

17. The implantable medical device of claim 1, wherein the drug delivery composition comprises a durable drug delivery composition comprising a bioactive agent in combination with a plurality of polymers, including a first polymer component comprising polyalkyl(meth)acrylate or aromatic poly(meth)acrylate and a second polymer component comprising poly(ethylene-co-vinyl acetate).

18. The implantable medical device of claim 17, wherein the first polymer component is selected from poly butyl(meth)acrylate, polyaryl(meth)acrylate, polyaralkyl(meth)acrylate, polyaryloxyalkyl(meth)acrylate, and combinations thereof.

19. The implantable medical device of claim 1, wherein the drug delivery composition comprises a biodegradable polymer selected from:
(a) a natural biodegradable polysaccharide selected from amylose, maltodextrin, amylopectin, starch, dextran, hyaluronic acid, heparin, chondroitin sulfate, dermatan sulfate, heparan sulfate, keratan sulfate, dextran sulfate, pentosan polysulfate, chitosan, and combinations thereof;
(b) a synthetic biodegradable polymer selected from polyanhydrides, polycaprolactone, polyglycolic acid, poly-L-lactic acid, poly-D-L-lactic, poly(D-lactic-co-glycolic acid), poly(D,L-lactic-co-glycolic acid), poly(ε-caprolactone), poly(lactic acid-co-lysine), poly(lactic acid-co-trimethylene carbonate), poly(valerolactone), poly(Hydroxyl butyrate), poly(hydrovalerate), polyphosphate esters, poly(hydroxybutyrate), polycarbonate, polyanhydride, poly(ortho esters), poly(phosphoesters), polyesters, polyamides, polyphosphazenes, poly(p-dioxane), poly(amino acid), polydioxanone, poly(propylene fumarate), poly(ethyleneoxide), poly(butyleneterephthalate), and combinations thereof; and
(c) combinations thereof.

20. A method of making an implantable medical device capable of delivering one or more biologically active agents to a patient:
obtaining a device that comprises one or more frictional surfaces;
forming one or more shallow recesses on one or more frictional surfaces; and
introducing a drug delivery composition into one or more shallow recesses.
